(19) United States
(12) Patent Application Publication
(10) Pub. No.: US 2006/0211733 A1
(43) Pub. Date: Sep. 21, 2006

(54) Methods of preventing and treating opioid bowel dysfunction

(75) Inventors: Lee Techner, Wynnewood, PA (US); Maryann Cherubini, Spring City, PA (US); Joseph F. Foss, Gates Mills, OH (US); Bruce A. Wallin, Haverford, PA (US); David Jackson, Cape Coral, FL (US); Wei Du, Norristown, PA (US); William K. Schmidt, Davis, CA (US); Virginia D. Schmith, Chapel Hill, NC (US); Jerry W. Snidow, Chapel Hill, NC (US); Eric R. Mortensen, Doylestown, PA (US)

Correspondence Address: WOODCOCK WASHBURN LLP, ONE LIBERTY PLACE, 46TH FLOOR, 1650 MARKET STREET, PHILADELPHIA, PA, 19103, US

(73) Assignees: Adolor Corporation, Exton, PA (US); Glaxo Group Limited, Greenford (GB)

(21) Appl. No.: 11/366,330

(22) Filed: Mar. 2, 2006

Related U.S. Application Data
(60) Provisional application No. 60/658,770, filed on Mar. 4, 2005.

(51) Int. Cl. A61K 31/445

(52) U.S. Cl. 514/317

(57) ABSTRACT

Methods of preventing and treating opioid bowel dysfunction are disclosed. In addition, methods of increasing the frequency of spontaneous complete bowel movements, methods for improving the quality of life of a patient suffering from opioid bowel dysfunction, methods for reducing a patient's dependence on laxatives, and methods for preventing or treating pain are also disclosed.

### CROSS REFERENCE TO RELATED APPLICATIONS

This application claims the benefit of U.S. Application No. 60/658,770 filed Mar. 4, 2005, the entire disclosure of which is incorporated herein by reference.

### FIELD OF THE INVENTION

The present invention generally relates to methods of preventing and treating opioid bowel dysfunction. More specifically, the present invention relates to methods of preventing and treating opioid bowel dysfunction in patients receiving long-term opioid treatment for pain.

### BACKGROUND OF THE INVENTION

Opioid bowel dysfunction is a reduction in frequency of bowel movements since beginning opioid therapy, generally less than about 3 spontaneous bowel movements per week along with difficulty in expelling stool, lumpy or hard stools, sensation of anorectal obstruction, or sensation of incomplete evacuation. Opioid bowel dysfunction may accompany the administration of opioid analgesics in patients, especially those patients receiving the long-term or chronic opioid analgesic treatment for pain. Because of this side effect, a patient may discontinue the pain medication or otherwise suffer from pain and discomfort associated with the inability to have spontaneous and/or complete bowel movements. Therefore, it would be desirable to provide methods for preventing and/or treating opioid bowel dysfunction in patients receiving opioid analgesics for pain while maintaining an acceptable pain tolerability profile. The methods of the present invention are directed toward these, as well as other, important ends.

### SUMMARY OF THE INVENTION

The methods of the present invention are directed to treating and preventing non-opioid induced gastrointestinal dysfunction, increasing the frequency of spontaneous complete bowel movements, improving the quality of life of a patient suffering from opioid bowel dysfunction, and methods for reducing a patient's dependence on laxatives. In addition, the methods of the present invention are directed to treating and preventing pain.

Accordingly, the present invention is directed, in part, to methods of treating or preventing opioid bowel dysfunction in a patient in need thereof, comprising the step of:

administering to said patient a dosage level of:

about 0.5 mg twice daily (BID);

about 1 mg once daily (QD); or

about 1 mg twice daily (BID);

preferably for about one week to about six weeks, of at least one 4-aryl-piperidine derivative or a stereoisomer, a prodrug, a pharmaceutically acceptable salt, a hydrate, a solvate, an acid salt hydrate, an N-oxide or an isomorphic form thereof;

wherein said patient is receiving chronic exogenous opioids for pain; and

wherein said 4-aryl-piperidine derivative is [[2-[[-4-(3-hydroxyphenyl)-3,4-dimethylpiperidin-1-yl]methyl]-3-phenylpropanoyl]amino]acetic acid or a compound of formula (IA): 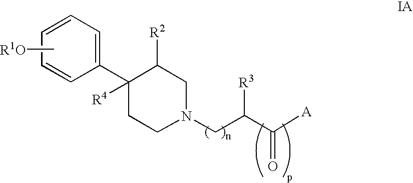
wherein:
R¹ is hydrogen or alkyl;
R² is hydrogen, alkyl or alkenyl;
R³ is hydrogen, alkyl, alkenyl, aryl, cycloalkyl, cycloalkenyl, cycloalkyl-substituted alkyl, cycloalkenyl-substituted alkyl or aralkyl;
R⁴ is hydrogen, alkyl or alkenyl;
A is OR⁵ or NR⁶R⁷;
R⁵ is hydrogen, alkyl, alkenyl, cycloalkyl, cycloalkenyl, cycloalkyl-substituted alkyl, cycloalkenyl-substituted alkyl, or aralkyl;
R⁶ is hydrogen or alkyl;
R⁷ is hydrogen, alkyl, alkenyl, cycloalkyl, aryl, cycloalkyl-substituted alkyl, cycloalkenyl, cycloalkenyl-substituted alkyl, aralkyl, B, or alkylene substituted B or, together with the nitrogen atom to which they are attached, R⁶ and R⁷ form a heterocyclic ring;
B is 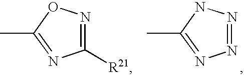
C(═O)W or NR⁸R⁹;
R⁸ is hydrogen or alkyl;
R⁹ is hydrogen, alkyl, alkenyl, cycloalkyl-substituted alkyl, cycloalkyl, cycloalkenyl, cycloalkenyl-substituted alkyl, aryl or aralkyl or, together with the nitrogen atom to which they are attached, R⁸ and R⁹ form a heterocyclic ring;
W is OR¹⁰, NR¹¹R¹², or OE;
R¹⁰ is hydrogen, alkyl, alkenyl, cycloalkyl, cycloalkenyl, cycloalkyl-substituted alkyl, cycloalkenyl-substituted alkyl, or aralkyl;
R¹¹ is hydrogen or alkyl;
R¹² is hydrogen, alkyl, alkenyl, aryl, cycloalkyl, cycloalkenyl, cycloalkyl-substituted alkyl, cycloalkenyl-substituted alkyl, aralkyl or alkylene substituted C(═O)Y or, together with the nitrogen atom to which they are attached, R¹¹ and R¹² form a heterocyclic ring;
E is

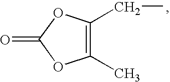

alkylene substituted (C═O)D, or —R¹³OC(═O)R¹⁴;
R¹³ is alkyl substituted alkylene;
R¹⁴ is alkyl;
D is OR¹⁵ or NR¹⁶R¹⁷;
R¹⁵ is hydrogen, alkyl, alkenyl, cycloalkyl, cycloalkenyl, cycloalkyl-substituted alkyl, cycloalkenyl-substituted alkyl, or aralkyl;

R¹⁶ is hydrogen, alkyl, alkenyl, aryl, aralkyl, cycloalkyl, cycloalkenyl, cycloalkyl-substituted alkyl or cycloalkenyl-substituted alkyl;
R¹⁷ is hydrogen or alkyl or, together with the nitrogen atom to which they are attached, R¹⁶ and R¹⁷ form a heterocyclic ring;
Y is OR¹⁸ or NR¹⁹R²⁰;
R¹⁸ is hydrogen, alkyl, alkenyl, cycloalkyl, cycloalkenyl, cycloalkyl-substituted alkyl, cycloalkenyl-substituted alkyl, or aralkyl;
R¹⁹ is hydrogen or alkyl;
R²⁰ is hydrogen, alkyl, alkenyl, aryl, cycloalkyl, cycloalkenyl, cycloalkyl-substituted alkyl, cycloalkenyl-substituted alkyl, or aralkyl or, together with the nitrogen atom to which they are attached, R¹⁹ and R²⁰ form a heterocyclic ring;
R²¹ is hydrogen or alkyl;
n is 0 to 4;
p is 0 or 1; and
provided that R¹⁰ is not hydrogen, when R¹ is hydrogen, R² is methyl, R³ is cycloalkyl-substituted alkyl, and R⁴ is methyl; and
provided that R¹⁰ is not alkyl, when R¹ is hydrogen, R² is methyl, R³ is aralkyl, and R⁴ is methyl.

In another embodiment, the invention is directed to methods of increasing the frequency of spontaneous complete bowel movements in a patient, comprising the step of:

administering to said patient a dosage level of:

about 0.5 mg twice daily (BID);

about 1 mg once daily (QD); or

about 1 mg twice daily (BID);

preferably for about one week to about six weeks, of at least one 4-aryl-piperidine derivative or a stereoisomer, a prodrug, a pharmaceutically acceptable salt, a hydrate, a solvate, an acid salt hydrate, an N-oxide or an isomorphic form thereof;

wherein said patient is receiving chronic exogenous opioids for pain; and

wherein said 4-aryl-piperidine derivative is [[2-[[-4-(3-hydroxyphenyl)-3,4-dimethylpiperidin-1-yl]methyl]-3-phenylpropanoyl]amino]acetic acid or a compound of formula (IA) as described herein.

In yet other embodiments, the invention is directed to methods for improving the quality of life of a patient suffering from opioid bowel dysfunction, comprising the step of:

administering to said patient a dosage level of:

about 0.5 mg twice daily (BID);

about 1 mg once daily (QD); or

about 1 mg twice daily (BID);

preferably for about one week to about six weeks, of at least one 4-aryl-piperidine derivative or a stereoisomer, a prodrug, a pharmaceutically acceptable salt, a hydrate, a solvate, an acid salt hydrate, an N-oxide or an isomorphic form thereof;

wherein said patient is receiving chronic exogenous opioids for pain; and

wherein said 4-aryl-piperidine derivative is [[2-[[-4-(3-hydroxyphenyl)-3,4-dimethylpiperidin-1-yl]methyl]-3-phenylpropanoyl]amino]acetic acid or a compound of formula (IA) as described herein.

In other embodiments, the invention is directed to methods for reducing a patient's dependence on laxatives, comprising the step of:

administering to said patient a dosage level of:

about 0.5 mg twice daily (BID);

about 1 mg once daily (QD); or

about 1 mg twice daily (BID);

preferably for about one week to about six weeks, of at least one 4-aryl-piperidine derivative or a stereoisomer, a prodrug, a pharmaceutically acceptable salt, a hydrate, a solvate, an acid salt hydrate, an N-oxide or an isomorphic form thereof;

wherein said patient is receiving chronic exogenous opioids for pain; and

wherein said 4-aryl-piperidine derivative is [[2-[[-4-(3-hydroxyphenyl)-3,4-dimethylpiperidin-1-yl]methyl]-3-phenylpropanoyl]amino]acetic acid or a compound of formula (IA) as described herein.

In still further embodiments, the invention is directed to methods for preventing or treating pain, comprising the step of:

administering to said patient at least one opioid; and

a dosage level of:

about 0.5 mg twice daily (BID);

about 1 mg once daily (QD); or

about 1 mg twice daily (BID);

preferably for about one week to about six weeks, of at least one 4-aryl-piperidine derivative or a stereoisomer, a prodrug, a pharmaceutically acceptable salt, a hydrate, a solvate, an acid salt hydrate, an N-oxide or an isomorphic form thereof;

wherein said 4-aryl-piperidine derivative is [[2-[[-4-(3-hydroxyphenyl)-3,4-dimethylpiperidin-1-yl]methyl]-3-phenylpropanoyl]amino]acetic acid or a compound of formula (IA) as described herein.

### BRIEF DESCRIPTION OF THE DRAWINGS

The invention is described with reference to the following figures:

FIG. 1 is a summary of the study design used in the examples.

FIG. 2 is a summary of the primary pain condition for the subjects in each cohort in the examples.

FIG. 3 is a summary of the opioid and pain history for the subjects in each cohort in the examples.

FIG. 4 shows the change in weekly spontaneous bowel movements (SBM) frequency for weeks 1 to 3 for the study described in the examples.

FIG. 5 shows the change in weekly spontaneous bowel movements (SBM) frequency for weeks 1 to 6 for the study described in the examples.

FIG. 6 is a plot of the average weekly SBM over time for the study described in the examples.

FIG. 7 shows the change in weekly spontaneous complete bowel movements (SCBM) frequency for weeks 1 to 3 for the study described in the examples.

FIG. 8 is a plot of the average weekly SCBM over time for the study described in the examples.

FIG. 9 shows the change in weekly complete bowel movements (CBM) frequency for weeks 1 to 3 for the study described in the examples.

FIG. 10 shows the change in weekly bowel movements (BM) frequency for weeks 1 to 3 for the study described in the examples.

FIG. 11 is a plot of the average weekly BM over time for the study for the study described in the examples.

FIG. 12 is the global improvement responder rate for weeks 1 to 3 for the study described in the examples.

FIG. 13 is a plot of the global improvement responders over time for the study described in the examples.

FIG. 14 are the global improvement scale ratings for the study described in the examples.

FIG. 15 is a plot of average daily straining over time for the study described in the examples.

FIG. 16 is a plot of the percentage of BMs with incomplete evacuation for the study described in the examples.

FIG. 17 a plot of responders with at least 3 SBMs per week for the study described in the examples.

FIG. 18 is a plot of responders with at least 3 SCBMs per week for the study described in the examples.

FIG. 19 is a plot of percentage of days with at least one SBM for the study described in the examples.

FIG. 20 is a chart of adverse events for each of the cohorts of the study for the study described in the examples.

FIG. 21 is a plot of average pain intensity scores over time for the study described in the examples.

FIG. 22 is a chart of the opioid total daily dose for each of the cohorts for the study described in the examples.

### DETAILED DESCRIPTION OF THE INVENTION

The methods of the present invention are directed to treating and preventing non-opioid induced gastrointestinal dysfunction, increasing the frequency of spontaneous complete bowel movements, improving the quality of life of a patient suffering from opioid bowel dysfunction, and methods for reducing a patient's dependence on laxatives, comprising the step of:

administering to said patient a dosage level of:

about 0.5 mg twice daily (BID);

about 1 mg once daily (QD); or

about 1 mg twice daily (BID);

preferably for about one week to about six weeks, of at least one 4-aryl-piperidine derivative or a stereoisomer, a prodrug, a pharmaceutically acceptable salt, a hydrate, a solvate, an acid salt hydrate, an N-oxide or an isomorphic form thereof;

wherein said patient is receiving chronic exogenous opioids for pain; and

wherein said 4-aryl-piperidine derivative is [[2-[[-4-(3-hydroxyphenyl)-3,4-dimethylpiperidin-1-yl]methyl]-3-phenylpropanoyl]amino]acetic acid or a compound of formula (IA) as described herein.

In certain embodiments, the methods are directed to treating and preventing non-opioid induced gastrointestinal dysfunction, increasing the frequency of spontaneous complete bowel movements, improving the quality of life of a patient suffering from opioid bowel dysfunction, and methods for reducing a patient's dependence on laxatives, comprising the step of:

administering to said patient a dosage level of:

no greater than about 5 mg twice daily (BID), preferably no greater than about 2 mg BID, more preferably no greater than about 1 mg BID, and even more preferably no greater than about 0.5 mg BID;

preferably for about one week to about six weeks, of at least one 4-aryl-piperidine derivative or a stereoisomer, a prodrug, a pharmaceutically acceptable salt, a hydrate, a solvate, an acid salt hydrate, an N-oxide or an isomorphic form thereof;

wherein said patient is receiving chronic exogenous opioids for pain; and

wherein said 4-aryl-piperidine derivative is [[2-[[-4-(3-hydroxyphenyl)-3,4-dimethylpiperidin-1-yl]methyl]-3-phenylpropanoyl]amino]acetic acid or a compound of formula (IA) as described herein.

In addition, the methods of the present invention are directed to preventing or treating pain, comprising the step of:

administering to said patient at least one opioid and a dosage level of:

about 0.5 mg twice daily (BID);

about 1 mg once daily (QD); or

about 1 mg twice daily (BID);

preferably for about one week to about six weeks, of at least one 4-aryl-piperidine derivative or a stereoisomer, a prodrug, a pharmaceutically acceptable salt, a hydrate, a solvate, an acid salt hydrate, an N-oxide or an isomorphic form thereof;

wherein said 4-aryl-piperidine derivative is [[2-[[-4-(3-hydroxyphenyl)-3,4-dimethylpiperidin-1-yl]methyl]-3-phenylpropanoyl]amino]acetic acid or a compound of formula (IA) as described herein.

In addition, the methods of the present invention are directed to preventing or treating pain, comprising the step of:

administering to said patient at least one opioid and a dosage level of:

no greater than about 5 mg twice daily (BID), preferably no greater than about 2 mg BID, more preferably no greater than about 1 mg BID, and even more preferably no greater than about 0.5 mg BID;

preferably for about one week to about six weeks, of at least one 4-aryl-piperidine derivative or a stereoisomer, a prodrug, a pharmaceutically acceptable salt, a hydrate, a solvate, an acid salt hydrate, an N-oxide or an isomorphic form thereof;

wherein said 4-aryl-piperidine derivative is [[2-[[-4-(3-hydroxyphenyl)-3,4-dimethylpiperidin-1-yl]methyl]-3″-phenylpropanoyl]amino]acetic acid or a compound of formula (IA) as described herein.

As employed above and throughout the disclosure, the following terms, unless otherwise indicated, shall be understood to have the following meanings.

As used herein, “alkyl” refers to an optionally substituted, saturated straight, branched, or cyclic hydrocarbon having from about 1 to about 20 carbon atoms (and all combinations and subcombinations of ranges and specific numbers of carbon atoms therein), with from about 1 to about 8 carbon atoms, herein referred to as “lower alkyl”, being preferred. “Branched” refers to an alkyl group in which a lower alkyl group, such as methyl, ethyl, or propyl, is attached to a linear alkyl chain. In certain preferred embodiments, the alkyl group is a C₁-C₅ alkyl group, i.e., a branched or linear alkyl group having from 1 to about 5 carbons. In other preferred embodiments, the alkyl group is a C₁-C₃ alkyl group, i.e., a branched or linear alkyl group having from 1 to about 3 carbons. Exemplary alkyl groups include methyl, ethyl, n-propyl, isopropyl, butyl, isobutyl, sec-butyl, tert-butyl, pentyl, hexyl, heptyl, octyl, nonyl, and decyl. “Lower alkyl” refers to an alkyl group having 1 to about 6 carbon atoms. Preferred alkyl groups include the lower alkyl groups of 1 to about 3 carbons. Alkyl groups include, but are not limited to, methyl, ethyl, n-propyl, isopropyl, n-butyl, isobutyl, t-butyl, n-pentyl, cyclopentyl, isopentyl, neopentyl, n-hexyl, isohexyl, cyclohexyl, cyclooctyl, adamantyl, 3-methylpentyl, 2,2-dimethylbutyl, and 2,3-dimethylbutyl.

As used herein, “alkylene” refers to a bivalent alkyl radical having the general formula —(CH₂)ₙ—, where n is 1 to 10, and all combinations and subcombinations of ranges therein. The alkylene group may be straight, branched or cyclic. Non-limiting examples include methylene, methylene (—CH₂—), ethylene (—CH₂CH₂—), propylene (—(CH₂)₃—), trimethylene, pentamethylene, and hexamethylene. There may be optionally inserted along the alkylene group one or more oxygen, sulfur or optionally substituted nitrogen atoms, wherein the nitrogen substituent is alkyl as described previously. Alkylene groups can be optionally substituted. The term “lower alkylene” herein refers to those alkylene groups having from about 1 to about 6 carbon atoms. Preferred alkylene groups have from about 1 to about 4 carbons.

As used herein, “aralkylene” refers to a bivalent alkyl radical having the general formula —(CH₂)ₙ—, wherein any one of the hydrogens on the alkylene radical is replaced by an aryl group, and where n is 1 to 10. Aralkylene groups can be optionally substituted. Non-limiting examples include phenylmethylene, 2-phenyltrimethylene, 3-(p-anisyl)-pentamethylene, and 2-(m-trifluromethylphenyl)-hexamethylene. Aralkylene groups can be substituted or unsubstituted. The term “lower aralkylene” herein refers to those aralkylene groups having from about 1 to about 6 carbon atoms in the alkylene portion of the aralkylene group.

As used herein, “alkenyl” refers to a monovalent alkyl radical containing at least one carbon-carbon double bond and having from 2 to about 10 carbon atoms in the chain, and all combinations and subcombinations of ranges therein. Alkenyl groups can be optionally substituted. In certain preferred embodiments, the alkenyl group is a C₂-C₁₀ alkyl group, i.e., a branched or linear alkenyl group having from 2 to about 10 carbons. In other preferred embodiments, the alkenyl group is a C₂-C₆ alkenyl group, i.e., a branched or linear alkenyl group having from 2 to about 6 carbons. In still other preferred embodiments, the alkenyl group is a C₃-C₁₀ alkenyl group, i.e., a branched or linear alkenyl group having from about 3 to about 10 carbons. In yet other preferred embodiments, the alkenyl group is a C₂-C₅ alkenyl group, i.e., a branched or linear alkenyl group having from 2 to about 5 carbons. Exemplary alkenyl groups include, for example, vinyl, propenyl, butenyl, pentenyl hexenyl, heptenyl, octenyl, nonenyl and decenyl groups.

As used herein, the term “alkenylene” refers to an alkylene group containing at least one carbon-carbon double bond. Exemplary alkenylene groups include, for example, ethenylene (—CH═CH—) and propenylene (—CH═CHCH₂—). Preferred alkenylene groups have from 2 to about 4 carbons.

As used herein, “aryl” refers to an optionally substituted, mono-, di-, tri-, or other multicyclic aromatic ring system having from about 5 to about 50 carbon atoms (and all combinations and subcombinations of ranges and specific numbers of carbon atoms therein), with from about 6 to about 10 carbons being preferred. Non-limiting examples include, for example, phenyl, naphthyl, anthracenyl, and phenanthrenyl.

As used herein, “aralkyl” refers to alkyl radicals bearing an aryl substituent and have from about 6 to about 50 carbon atoms (and all combinations and subcombinations of ranges and specific numbers of carbon atoms therein), with from about 6 to about 10 carbon atoms being preferred. Aralkyl groups can be optionally substituted in either the aryl or alkyl portions. Non-limiting examples include, for example, phenylmethyl(benzyl), diphenylmethyl, triphenylmethyl, phenylethyl, diphenylethyl and 3-(4-methylphenyl)propyl.

As used herein, “heteroaryl” refers to an optionally substituted, mono-, di-, tri-, or other multicyclic aromatic ring system that includes at least one, and preferably from 1 to about 4 sulfur, oxygen, or nitrogen heteroatom ring members. Heteroaryl groups can have, for example, from about 3 to about 50 carbon atoms (and all combinations and subcombinations of ranges and specific numbers of carbon atoms therein), with from about 4 to about 10 carbons being preferred. Non-limiting examples of heteroaryl groups include, for example, pyrryl, furyl, pyridyl, 1,2,4-thiadiazolyl, pyrimidyl, thienyl, isothiazolyl, imidazolyl, tetrazolyl, pyrazinyl, pyrimidyl, quinolyl, isoquinolyl, thiophenyl, benzothienyl, isobenzofuryl, pyrazolyl, indolyl, purinyl, carbazolyl, benzimidazolyl, and isoxazolyl.

As used herein, “cycloalkyl” refers to an optionally substituted, alkyl group having one or more rings in their structures having from about 3 to about 20 carbon atoms (and all combinations and subcombinations of ranges and specific numbers of carbon atoms therein), with from about 3 to about 10 carbon atoms being preferred, with from about 3 to about 8 carbon atoms being more preferred, with from about 3 to about 6 carbon atoms being even more preferred. Multi-ring structures may be bridged or fused ring structures. The cycloalkyl group may be optionally substituted with, for example, alkyl, preferably C₁-C₃ alkyl, alkoxy, preferably C₁-C₃ alkoxy, or halo. Non-limiting examples include, for example, cyclopropyl, cyclobutyl, cyclopentyl, cyclohexyl, cycloheptyl cyclooctyl, and adamantyl.

As used herein, “cycloalkyl-substituted alkyl” refers to a linear alkyl group, preferably a lower alkyl group, substituted at a terminal carbon with a cycloalkyl group, preferably a C₃-C₈ cycloalkyl group. Non-limiting examples include, for example, cyclohexylmethyl, cyclohexylethyl, cyclopentylethyl, cyclopentylpropyl, cyclopropylmethyl and the like.

As used herein, “cycloalkenyl” refers to an olefinically unsaturated cycloalkyl group having from about 4 to about 10 carbons, and all combinations and subcombinations of ranges therein. In preferred embodiments, the cycloalkenyl group is a C₅-C₈ cycloalkenyl group, i.e., a cycloalkenyl group having from about 5 to about 8 carbons.

As used herein, “alkylcycloalkyl” refers to an optionally substituted ring system comprising a cycloalkyl group having one or more alkyl substituents. Non-limiting examples include, for example, alkylcycloalkyl groups include 2-methylcyclohexyl, 3,3-dimethylcyclopentyl, trans-2,3-dimethylcyclooctyl, and 4-methyldecahydronaphthalenyl.

As used herein, “heteroaralkyl” refers to an optionally substituted, heteroaryl substituted alkyl radicals having from about 2 to about 50 carbon atoms (and all combinations and subcombinations of ranges and specific numbers of carbon atoms therein), with from about 6 to about 25 carbon atoms being preferred. Non-limiting examples include 2-(1H-pyrrol-3-yl)ethyl, 3-pyridylmethyl, 5-(2H-tetrazolyl)methyl, and 3-(pyrimidin-2-yl)-2-methylcyclopentanyl.

As used herein, “heterocycloalkyl” refers to an optionally substituted, mono-, di-, tri-, or other multicyclic aliphatic ring system that includes at least one, and preferably from 1 to about 4 sulfur, oxygen, or nitrogen heteroatom ring members. Heterocycloalkyl groups can have from about 3 to about 20 carbon atoms (and all combinations and subcombinations of ranges and specific numbers of carbon atoms therein), with from about 4 to about 10 carbons being preferred. The heterocycloalkyl group may be unsaturated, and may also be fused to aromatic rings. Non-limiting examples include, for example, tetrahydrofuranyl, tetrahydrothienyl, piperidinyl, pyrrolidinyl, isoxazolidinyl, isothiazolidinyl, pyrazolidinyl, oxazolidinyl, thiazolidinyl, piperazinyl, morpholinyl, piperadinyl, decahydroquinolyl, octahydrochromenyl, octahydro-cyclopenta[c]pyranyl, 1,2,3,4,-tetrahydroquinolyl, octahydro-[2]pyrindinyl, decahydro-cycloocta[c]furanyl, and imidazolidinyl.

As used herein, the term “spiroalkyl” refers to an optionally substituted, alkylene diradical, both ends of which are bonded to the same carbon atom of the parent group to form a spirocyclic group. The spiroalkyl group, taken together with its parent group, as herein defined, has 3 to 20 ring atoms. Preferably, it has 3 to 10 ring atoms. Non-limiting examples of a spiroalkyl group taken together with its parent group include 1-(1-methyl-cyclopropyl)-propan-2-one, 2-(1-phenoxy-cyclopropyl)-ethylamine, and 1-methyl-spiro[4.7]dodecane.

As used herein, the term “alkoxy” refers to an optionally substituted alkyl-O— group wherein alkyl is as previously defined. Non-limiting examples include, for example, include methoxy, ethoxy, n-propoxy, i-propoxy, n-butoxy, and heptoxy.

As used herein, the term “aryloxy” refers to an optionally substituted aryl-O— group wherein aryl is as previously defined. Non-limiting examples include, for example, phenoxy and naphthoxy.

As used herein, the term “aralkoxy” refers to an optionally substituted aralkyl-O— group wherein aralkyl is as previously defined. Non-limiting examples include, for example, benzyloxy, 1-phenylethoxy, 2-phenylethoxy, and 3-naphthylheptoxy.

As used herein, the term “aryloxyaryl” refers to an aryl group with an aryloxy substituent wherein aryloxy and aryl are as previously defined. Aryloxyaryl groups can be optionally substituted. Non-limiting examples include, for example, phenoxyphenyl, and naphthoxyphenyl.

As used herein, the term “heteroarylaryl” refers to an aryl group with a heteroaryl substituent wherein heteroaryl and aryl are as previously defined. Heteroarylaryl groups can be optionally substituted. Non-limiting examples include, for example, 3-pyridylphenyl, 2-quinolylnaphthalenyl, and 2-pyrrolylphenyl.

As used herein, the term “alkoxyaryl” refers to an aryl group bearing an alkoxy substituent wherein alkoxy and aryl are as previously defined. Alkoxyaryl groups can be optionally substituted. Non-limiting examples include, for example, para-anisyl, meta-t-butoxyphenyl, and methylendioxyphenyl.

As used herein, the term “carbon chain of said alkoxy interrupted by a nitrogen atom” refers to a carbon chain of an alkoxy group, wherein a nitrogen atom has been inserted between two adjacent carbon atoms of the carbon chain and wherein alkoxy is as previously defined. Both the alkoxy group and the nitrogen atom can be optionally substituted. Exemplary groups include —OCH₂CH₂N(CH₃)CH₂CH₃ and —OCH₂CH₂NHCH₃.

As used herein, the term “heterocycloalkylheteroaryl” refers to an heteroaryl group with a heterocycloalkyl substituent wherein heterocycloalkyl and heteroaryl are as previously defined. Heterocycloalkylheteroaryl groups can be optionally substituted. Exemplary heterocycloalkylheteroaryl groups include 3-[N-morpholinyl]pyridine and 3-[2-piperidinyl]pyridine.

As used herein, the term “heteroarylheteroaryl” refers to a heteroaryl group with a heteroaryl substituent wherein heteroaryl is as previously defined. Heteroarylherteroaryl groups can be optionally substituted. Exemplary heteroarylheteroaryl groups include 4-[3-pyridyl]pyridine and 2-[2-quinolyl]quinuclidine.

As used herein, the term “aralkoxyaryl” refers to an aryl group with an aralkoxy substituent wherein aralkoxy and aryl are as previously defined. Aralkoxyaryl groups can be optionally substituted. Exemplary aralkoxyaryl groups include benzyloxyphenyl and meta-toluenyloxyphenyl.

As used herein, the term “arylheteroaryl” refers to a heteroaryl group with an aryl substituent wherein aryl and heteroaryl are as previously defined. Arylheteroaryl groups can be optionally substituted. Exemplary arylheteroaryl groups include 3-phenylpyridyl and 2-naphthalenylquinolinyl.

As used herein, the term “alkoxyheteroaryl” refers to an heteroaryl group with an alkoxy substituent wherein alkoxy and heteroaryl are as previously defined. Alkoxyheteroaryl groups can be optionally substituted. Exemplary alkoxyheteroaryl groups include 2-methoxypyridine and 6-n-propoxyquinoline.

As used herein, “bicycloalkyl” refers to an optionally substituted, alkyl group having two bridged rings in its structure and having from about 7 to about 20 carbon atoms (and all combinations and subcombinations of ranges and specific numbers of carbon atoms therein), with from about 7 to about 15 carbon atoms being preferred. Exemplary bicycloalkyl-ring structures include, but are not limited to, norbornyl, bornyl, [2.2.2]-bicyclooctyl, cis-pinanyl, trans-pinanyl, camphanyl, iso-bornyl, and fenchyl.

As used herein, “bicycloalkenyl” refers to an optionally substituted, alkenyl group having two bridged rings in its structure and having from about 7 to about 20 carbon atoms (and all combinations and subcombinations of ranges and specific numbers of carbon atoms therein), with from about 7 to about 15 carbon atoms being preferred. Exemplary bicycloalkenyl-ring structures include, but are not limited to, bicyclo[2.2.1]hept-5-en-2-yl, bornenyl, [2.2.2]-bicyclooct-5-en-2-yl, α-pinenyl, β-pinenyl, camphenyl, and fenchyl.

As used herein, “carboxy” refers to a —C(═O)OH group.

As used herein, “alkanoyl” refers to a —C(═O)-alkyl group, wherein alkyl is as previously defined. Exemplary alkanoyl groups include acetyl(ethanoyl), n-propanoyl, n-butanoyl, 2-methylpropanoyl, n-pentanoyl, 2-methylbutanoyl, 3-methylbutanoyl, 2,2-dimethylpropanoyl, heptanoyl, decanoyl, and palmitoyl.

As used herein, “alkoxy-alkyl” refers to an alkyl-O-alkyl group where alkyl is as previously described.

As used herein, “heterocyclic” refers to a monocyclic or multicyclic ring system carbocyclic radical containing from about 4 to about 10 members, and all combinations and subcombinations of ranges therein, wherein one or more of the members is an element other than carbon, for example, nitrogen, oxygen or sulfur. The heterocyclic group may be aromatic or nonaromatic. Non-limiting examples include, for example, pyrrole and piperidine groups.

As used herein, “halo” refers to fluoro, chloro or bromo.

Typically, substituted chemical moieties include one or more substituents that replace hydrogen. Exemplary substituents include, for example, halo (e.g., F, Cl, Br, I), alkyl, cycloalkyl, alkylcycloalkyl, alkenyl, alkynyl, aralkyl, aryl, heteroaryl, heteroaralkyl, spiroalkyl, heterocycloalkyl, hydroxyl (—OH), nitro (—NO₂), cyano (—CN), amino (—NH₂), —N— substituted amino (—NHR″), —N,N-disubstituted amino (—N(R″)R″), carboxyl (—COOH), —C(═O)R″, —OR″, —C(═O)OR″, —NHC(═O)R″, aminocarbonyl (—C(═O)NH₂), —N-substituted aminocarbonyl (—C(═O)NHR″), -N,N-disubstituted aminocarbonyl (—C(═O)N(R″)R″), thiol, thiolato (SR″), sulfonic acid (SO₃H), phosphonic acid (PO₃H), S(═O)₂R″, S(═O)₂NH₂, S(═O)₂ NHR″, S(═O)₂NR″R″, NHS(═O)₂R″, NR″S(═O)₂R″, CF₃, CF₂CF₃, NHC(═O)NHR″, NHC(═O)NR″R″, NR″C(═O)NHR″, NR″C(═O)NR″R″, NR″C(═O)R″ and the like. In relation to the aforementioned substituents, each moiety R″ can be, independently, any of H, alkyl, cycloalkyl, alkenyl, aryl, aralkyl, heteroaryl, or heterocycloalkyl, for example.

As used herein, the term “opioid bowel dysfunction” refers to a reduction in frequency of bowel movements since beginning opioid therapy, generally less than about 3 spontaneous bowel movements per week along with one of more of difficulty in expelling stool, lumpy or hard stools, sensation of anorectal obstruction, or sensation of incomplete evacuation.

As used herein, the term “pain” includes both cancer pain and “non-malignant pain,” which includes pain not caused by cancer and includes, but is not limited to, back pain, degenerative disk disease, failed back syndrome, complex regional pain syndrome, fibromyalgia, arthritis, headache, and combinations thereof.

As used herein, the phrase “patient is receiving chronic exogenous opioids” refers to a patient receiving a daily dose of at least about 30 mg of an oral morphine equivalent for at least about 1 month, by any route of administration

As used herein, the phrase “quality of life” refers to an improvement in the Jansen PAC-SYS descriptors, including abdominal symptoms, rectal symptoms, and stool symptoms (such as incomplete bowel movement, bowel movements that were too hard, bowel movements that were too small, straining or squeezing to try to pass bowel movements, and false urge to defecate).

As used herein, the term “chronic constipation” refers to a low defecation rate of about three or fewer bowel movements in about a 7-day period with no known cause or etiology.

As used herein, the term “low stool frequency” refers to a defecation rate of about three or fewer bowel movements in about a 7-day period.

As used herein, the term “poor stool consistency” refers to hard, lumpy stools (Bristol Stool Form Scale type 1 or 2; Heaton, et al., Gut, 1991, 73-79).

As used herein, “dosage unit” refers to physically discrete units suited as unitary dosages for the particular individual to be treated. Each unit may contain a predetermined quantity of active compound(s) calculated to produce the desired therapeutic effect(s) in association with the required pharmaceutical carrier. The specification for the dosage unit forms of the invention may be dictated by (a) the unique characteristics of the active compound(s) and the particular therapeutic effect(s) to be achieved, and (b) the limitations inherent in the art of compounding such active compound(s).

As used herein, “pharmaceutically acceptable” refers to those compounds, materials, compositions, and/or dosage forms that are, within the scope of sound medical judgment, suitable for contact with the tissues of human beings and animals without excessive toxicity, irritation, allergic response, or other problem complications commensurate with a reasonable benefit/risk ratio.

As used herein, “pharmaceutically acceptable salts” refer to derivatives of the disclosed compounds wherein the parent compound is modified by making acid or base salts thereof. Examples of pharmaceutically acceptable salts include, but are not limited to, mineral or organic acid salts of basic residues such as amines; alkali or organic salts of acidic residues such as carboxylic acids; and the like. The pharmaceutically acceptable salts include the conventional non-toxic salts or the quaternary ammonium salts of the parent compound formed, for example, from non-toxic inorganic or organic acids. For example, such conventional non-toxic salts include those derived from inorganic acids such as hydrochloric, hydrobromic, sulfuric, sulfamic, phosphoric, nitric and the like; and the salts prepared from organic acids such as acetic, propionic, succinic, glycolic, stearic, lactic, malic, tartaric, citric, ascorbic, pamoic, maleic, hydroxymaleic, phenylacetic, glutamic, benzoic, salicylic, sulfanilic, 2-acetoxybenzoic, fumaric, toluenesulfonic, methanesulfonic, ethane disulfonic, oxalic, isethionic, and the like. These physiologically acceptable salts are prepared by methods known in the art, e.g., by dissolving the free amine bases with an excess of the acid in aqueous alcohol, or neutralizing a free carboxylic acid with an alkali metal base such as a hydroxide, or with an amine.

Compounds described herein throughout, can be used or prepared in alternate forms. For example, many amino-containing compounds can be used or prepared as an acid addition salt. Often such salts improve isolation and handling properties of the compound. For example, depending on the reagents, reaction conditions and the like, compounds as described herein can be used or prepared, for example, as their hydrochloride or tosylate salts. Isomorphic forms, all chiral and racemic forms, N-oxide, hydrates, solvates, and acid salt hydrates, are also contemplated to be within the scope of the present invention.

Certain acidic or basic compounds of the present invention may exist as zwitterions. All forms of the compounds, including free acid, free base and zwitterions, are contemplated to be within the scope of the present invention. It is well known in the art that compounds containing both amino and carboxyl groups often exist in equilibrium with their zwitterionic forms. Thus, any of the compounds described herein throughout that contain, for example, both amino and carboxyl groups, also include reference to their corresponding zwitterions.

As used herein, “patient” refers to animals, including mammals, preferably humans.

As used herein, “prodrug” refers to compounds specifically designed to maximize the amount of active species that reaches the desired site of reaction that are of themselves typically inactive or minimally active for the activity desired, but through biotransformation are converted into biologically active metabolites.

As used herein, “stereoisomers” refers to compounds that have identical chemical constitution, but differ as regards the arrangement of the atoms or groups in space.

As used herein, “N-oxide” refers to compounds wherein the basic nitrogen atom of either a heteroaromatic ring or tertiary amine is oxidized to give a quaternary nitrogen bearing a positive formal charge and an attached oxygen atom bearing a negative formal charge.

As used herein, “hydrate” refers to a compound of the present invention which is associated with water in the molecular form, i.e., in which the H—OH bond is not split, and may be represented, for example, by the formula R.H₂O, where R is a compound of the invention. A given compound may form more than one hydrate including, for example, monohydrates (R.H₂O) or polyhydrates (R.nH₂O wherein n is an integer >1) including, for example, dihydrates (R.2H₂O), trihydrates (R.3H₂O), and the like, or hemihydrates, such as, for example, R.n/2H₂O, R.n/3H₂O, R.n/4H₂O and the like wherein n is an integer.

As used herein, “solvate” refers to a compound of the present invention which is associated with solvent in the molecular form, i.e., in which the solvent is coordinatively bound, and may be represented, for example, by the formula R.(solvent), where R is a compound of the invention. A given compound may form more than one solvate including, for example, monosolvates (R.(solvent)) or polysolvates (R.n(solvent)) wherein n is an integer >1) including, for example, disolvates (R.2(solvent)), trisolvates (R.3(solvent)), and the like, or hemisolvates, such as, for example, R.n/2(solvent), R.n/3(solvent), R.n/4(solvent) and the like wherein n is an integer. Solvents herein include mixed solvents, for example, methanol/water, and as such, the solvates may incorporate one or more solvents within the solvate.

As used herein, “acid salt hydrate” refers to a complex that may be formed through association of a compound having one or more base moieties with at least one compound having one or more acid moieties or through association of a compound having one or more acid moieties with at least one compound having one or more base moieties, said complex being further associated with water molecules so as to form a hydrate, wherein said hydrate is as previously defined and R represents the complex herein described above.

When any variable occurs more than one time in any constituent or in any formula, its definition in each occurrence is independent of its definition at every other occurrence. Combinations of substituents and/or variables are permissible only if such combinations result in stable compounds.

The piperidines derivatives useful in the methods of the invention as illustrated in formula (IA) can occur as the trans and cis stereochemical isomers at the 3- and 4-positions of the piperidine ring. The term “trans” as used herein refers, for example, in formula (IA) to the R² substituent being on the opposite side of the R⁴ substituent, whereas in the “cis” isomer, the R² substituent and the R⁴ substituent are on the same side of the ring. The present invention contemplates the individual stereoisomers, as well as racemic mixtures. In the most preferred compounds of formula (IA), the R² substituent and the R⁴ substituent are in the “trans” orientation on the piperidine.

In addition to the “cis” and “trans” orientation of the R² substituent and the R⁴ substituent of formula (IA), the absolute stereochemistry of the carbon atoms bearing R² substituent and the R⁴ substituent of formula (IA) is also defined as using the commonly employed “R” and “S” definitions (Orchin et al., The Vocabulary of Organic Chemistry, John Wiley and Sons, Inc., 1981, page 126, which is incorporated herein by reference). The preferred compounds of the present invention are in which the configuration of both the R² substituent and the R⁴ substituents of formula (IA) on the piperidine ring are “R.”

Furthermore, asymmetric carbon atoms may be introduced into the molecule depending on the structure of R⁴. As such, these classes of compounds can exist as the individual “R” or “S” stereoisomers at these chiral centers, or the racemic mixture of the isomers, and all are contemplated as within the scope of the present invention. Preferably, a substantially pure stereoisomer of the compounds of this invention is used, i.e., an isomer in which the configuration at the chiral center is “R” or “S”, i.e., those compounds in which the configuration at the three chiral centers are preferably 3R, 4R, S or 3R, 4R, R.

As used herein, “peripheral” or “peripherally-acting” refers to an agent that acts outside of the central nervous system.

As used herein, “centrally-acting” refers to an agent that acts within the central nervous system (CNS).

In certain preferred embodiments, the methods may involve a peripheral opioid antagonist compound. The term “peripheral” designates that the compound acts primarily on physiological systems and components external to the central nervous system. In preferred form, the peripheral opioid antagonist compounds employed in the methods of the present invention exhibit high levels of activity with respect to peripheral tissue, such as, gastrointestinal tissue, while exhibiting reduced, and preferably substantially no, CNS activity. The phrase “substantially no CNS activity,” as used herein, means that less than about 20% of the pharmacological activity of the compounds employed in the present methods is exhibited in the CNS, preferably less than about 15%, more preferably less than about 10%, even more preferably less than about 5% and most preferably 0% of the pharmacological activity of the compounds employed in the present methods is exhibited in the CNS.

Furthermore, it is preferred in certain embodiments of the invention that the compound of formula (IA) does not substantially cross the blood-brain barrier and thereby interfere with the receptors in the CNS. The phrase “does not substantially cross,” as used herein, means that less than about 20% by weight of the compound employed in the present methods crosses the blood-brain barrier, preferably less than about 15% by weight, more preferably less than about 10% by weight, even more preferably less than about 5% by weight and most preferably 0% by weight of the compound crosses the blood-brain barrier.

The methods of the present invention are directed to treating and preventing non-opioid induced gastrointestinal dysfunction, increasing the frequency of spontaneous complete bowel movements, improving the quality of life of a patient suffering from opioid bowel dysfunction, and methods for reducing a patient's dependence on laxatives. In other embodiments, the methods of the invention are directed to preventing or treating pain.

The methods of the present invention may further employ one or more other active ingredients that may be conventionally employed in preventing or treating gastrointestinal dysfunction. Such conventional ingredients include, for example, laxatives, fiber, stool softeners, or bowel stimulants. Typical or conventional ingredients that may be included in the opioid component are described, for example, in the Physicians' Desk Reference, 2003, the disclosure of which is hereby incorporated herein by reference, in its entirety. Other optional components that may be employed in the methods and compositions of the present invention, in addition to those exemplified above, would be readily apparent to one of ordinary skill in the art, once armed with the teachings of the present disclosure.

Suitable 4-aryl-piperidine derivatives and a stereoisomer, a prodrug, a pharmaceutically acceptable salt, a hydrate, a solvate, an acid salt hydrate, an N-oxide and an isomorphic form thereof. Preferred 4-aryl-piperidine derivatives include, for example, the compounds disclosed in U.S. Pat. No. 5,250,542; U.S. Pat. No. 5,159,081; U.S. Pat. No. 5,270,328; and U.S. Pat. No. 5,434,171, U.S. Pat. No. 6,451,806 and U.S. Pat. No. 6,469,030, the disclosures of which are hereby incorporated herein by reference, in their entireties.

Accordingly, the present invention is directed, in part, to methods of treating or preventing opioid bowel dysfunction in a patient in need thereof, comprising the step of:

administering to said patient a dosage level of:

about 0.5 mg twice daily (BID);

about 1 mg once daily (QD); or

about 1 mg twice daily (BID);

preferably for about one week to about six weeks, of at least one 4-aryl-piperidine derivative or a stereoisomer, a prodrug, a pharmaceutically acceptable salt, a hydrate, a solvate, an acid salt hydrate, an N-oxide or an isomorphic form thereof;

wherein said patient is receiving chronic exogenous opioids for pain; and

wherein said 4-aryl-piperidine derivative is [[2-[[-4-(3-hydroxyphenyl)-3,4-dimethylpiperidin-1-yl]methyl]-3-phenylpropanoyl]amino]acetic acid or a compound of formula (IA): 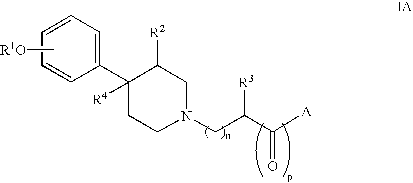
wherein:
R¹ is hydrogen or alkyl;
R² is hydrogen, alkyl or alkenyl;
R³ is hydrogen, alkyl, alkenyl, aryl, cycloalkyl, cycloalkenyl, cycloalkyl-substituted alkyl, cycloalkenyl-substituted alkyl or aralkyl;
R⁴ is hydrogen, alkyl or alkenyl;
A is OR⁵ or NR⁶R⁷;
R⁵ is hydrogen, alkyl, alkenyl, cycloalkyl, cycloalkenyl, cycloalkyl-substituted alkyl, cycloalkenyl-substituted alkyl, or aralkyl;
R⁶ is hydrogen or alkyl;
R⁷ is hydrogen, alkyl, alkenyl, cycloalkyl, aryl, cycloalkyl-substituted alkyl, cycloalkenyl, cycloalkenyl-substituted alkyl, aralkyl, B, or alkylene substituted B or, together with the nitrogen atom to which they are attached, R⁶ and R⁷ form a heterocyclic ring;
B is 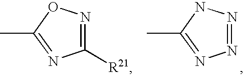
C(═O)W or NR⁸R⁹;
R⁸ is hydrogen or alkyl;
R⁹ is hydrogen, alkyl, alkenyl, cycloalkyl-substituted alkyl, cycloalkyl, cycloalkenyl, cycloalkenyl-substituted alkyl, aryl or aralkyl or, together with the nitrogen atom to which they are attached, R⁸ and R⁹ form a heterocyclic ring;
W is OR¹⁰, NR¹¹R¹², or OE;
R¹⁰ is hydrogen, alkyl, alkenyl, cycloalkyl, cycloalkenyl, cycloalkyl-substituted alkyl, cycloalkenyl-substituted alkyl, or aralkyl;
R¹¹ is hydrogen or alkyl;
R¹² is hydrogen, alkyl, alkenyl, aryl, cycloalkyl, cycloalkenyl, cycloalkyl-substituted alkyl, cycloalkenyl-substituted alkyl, aralkyl or alkylene substituted C(═O)Y or, together with the nitrogen atom to which they are attached, R¹¹ and R¹² form a heterocyclic ring;
E is

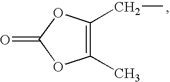

alkylene substituted (C═O)D, or —R¹³OC(═O)R¹⁴;
R¹³ is alkyl substituted alkylene;
R¹⁴ is alkyl;
D is OR¹⁵ or NR¹⁶R¹⁷;
R¹⁵ is hydrogen, alkyl, alkenyl, cycloalkyl, cycloalkenyl, cycloalkyl-substituted alkyl, cycloalkenyl-substituted alkyl, or aralkyl;
R¹⁶ is hydrogen, alkyl, alkenyl, aryl, aralkyl, cycloalkyl, cycloalkenyl, cycloalkyl-substituted alkyl or cycloalkenyl-substituted alkyl;
R¹⁷ is hydrogen or alkyl or, together with the nitrogen atom to which they are attached, R¹⁶ and R¹⁷ form a heterocyclic ring;
Y is OR¹⁸ or NR¹⁹R²⁰;
R¹⁸ is hydrogen, alkyl, alkenyl, cycloalkyl, cycloalkenyl, cycloalkyl-substituted alkyl, cycloalkenyl-substituted alkyl, or aralkyl;
R¹⁹ is hydrogen or alkyl;
R²⁰ is hydrogen, alkyl, alkenyl, aryl, cycloalkyl, cycloalkenyl, cycloalkyl-substituted alkyl, cycloalkenyl-substituted alkyl, or aralkyl or, together with the nitrogen atom to which they are attached, R¹⁹ and R²⁰ form a heterocyclic ring;
R²¹ is hydrogen or alkyl;
n is 0 to 4;
p is 0 or 1; and
provided that R¹⁰ is not hydrogen, when R¹ is hydrogen, R² is methyl, R³ is cycloalkyl-substituted alkyl, and R⁴ is methyl; and
provided that R¹⁰ is not alkyl, when R¹ is hydrogen, R² is methyl, R³ is aralkyl, and R⁴ is methyl.

In another embodiment, the invention is directed to methods of increasing the frequency of spontaneous complete bowel movements in a patient, comprising the step of:

administering to said patient a dosage level of:

about 0.5 mg twice daily (BID);

about 1 mg once daily (QD); or

about 1 mg twice daily (BID);

preferably for about one week to about six weeks, of at least one 4-aryl-piperidine derivative or a stereoisomer, a prodrug, a pharmaceutically acceptable salt, a hydrate, a solvate, an acid salt hydrate, an N-oxide or an isomorphic form thereof;

wherein said patient is receiving chronic exogenous opioids for pain; and

wherein said 4-aryl-piperidine derivative is [[2-[[-4-(3-hydroxyphenyl)-3,4-dimethylpiperidin-1-yl]methyl]-3-phenylpropanoyl]amino]acetic acid or a compound of formula (IA) as described herein.

In yet other embodiments, the invention is directed to methods for improving the quality of life of a patient suffering from opioid bowel dysfunction, comprising the step of:

administering to said patient a dosage level of:

about 0.5 mg twice daily (BID);

about 1 mg once daily (QD); or

about 1 mg twice daily (BID);

preferably for about one week to about six weeks, of at least one 4-aryl-piperidine derivative or a stereoisomer, a prodrug, a pharmaceutically acceptable salt, a hydrate, a solvate, an acid salt hydrate, an N-oxide or an isomorphic form thereof;

wherein said patient is receiving chronic exogenous opioids for pain; and

wherein said 4-aryl-piperidine derivative is [[2-[[-4-(3-hydroxyphenyl)-3,4-dimethylpiperidin-1-yl]methyl]-3-phenylpropanoyl]amino]acetic acid or a compound of formula (IA) as described herein.

In other embodiments, the invention is directed to methods for reducing a patient's dependence on laxatives, comprising the step of:

administering to said patient a dosage level of:

about 0.5 mg twice daily (BID);

about 1 mg once daily (QD); or

about 1 mg twice daily (BID);

preferably for about one week to about six weeks, of at least one 4-aryl-piperidine derivative or a stereoisomer, a prodrug, a pharmaceutically acceptable salt, a hydrate, a solvate, an acid salt hydrate, an N-oxide or an isomorphic form thereof;

wherein said patient is receiving chronic exogenous opioids for pain; and

wherein said 4-aryl-piperidine derivative is [[2-[[-4-(3-hydroxyphenyl)-3,4-dimethylpiperidin-1-yl]methyl]-3-phenylpropanoyl]amino]acetic acid or a compound of formula (IA) as described herein.

In still further embodiments, the invention is directed to methods for preventing or treating pain, comprising the step of:

administering to said patient at least one opioid and a dosage level of:

about 0.5 mg twice daily (BID);

about 1 mg once daily (QD); or

about 1 mg twice daily (BID);

preferably for about one week to about six weeks, of at least one 4-aryl-piperidine derivative or a stereoisomer, a prodrug, a pharmaceutically acceptable salt, a hydrate, a solvate, an acid salt hydrate, an N-oxide or an isomorphic form thereof;

wherein said 4-aryl-piperidine derivative is [[2-[[-4-(3-hydroxyphenyl)-3,4-dimethylpiperidin-1-yl]methyl]-3-phenylpropanoyl]amino]acetic acid or a compound of formula (IA) as described herein.

Suitable opioids include alfentanil, buprenorphine, butorphanol, codeine, dezocine, dihydrocodeine, fentanyl, hydrocodone, hydromorphone, levorphanol, meperidine (pethidine), methadone, morphine, nalbuphine, oxycodone, oxymorphone, pentazocine, propiram, propoxyphene, sufentanil, and tramadol. Preferred opioids include morphine, codeine, oxycodone, hydrocodone, dihydrocodeine, propoxyphene, fentanyl, and tramadol.

The opioid component of the present compositions may further include one or more other active ingredients that may be conventionally employed in analgesic and/or cough-cold-antitussive combination products. Such conventional ingredients include, for example, aspirin, acetaminophen, phenylpropanolamine, phenylephrine, chlorpheniramine, caffeine, and/or guaifenesin. Typical or conventional ingredients that may be included in the opioid component are described, for example, in the Physicians' Desk Reference, 1999, the disclosure of which is hereby incorporated herein by reference, in its entirety.

In addition, the opioid component may further include one or more compounds that may be designed to enhance the analgesic potency of the opioid and/or to reduce analgesic tolerance development. Such compounds include, for example, dextromethorphan or other NMDA antagonists (Mao, M. J. et al., Pain 1996, 67, 361), L-364,718 and other CCK antagonists (Dourish, C. T. et al., Eur. J. Pharmacol., 1988, 147, 469), NOS inhibitors (Bhargava, H. N. et al., Neuropeptides, 1996, 30, 219), PKC inhibitors (Bilsky, E. J. et al., J. Pharmacol. Exp. Ther. 1996, 277, 484), and dynorphin antagonists or antisera (Nichols, M. L. et al., Pain, 1997, 69, 317). The disclosures of each of the foregoing documents are hereby incorporated herein by reference, in their entireties.

In any of the methods described above, the compound may be stepped-up in increments to reach the desired dosage level over a certain duration, preferably over about 1 to about 5 days, more preferably over about 1 to 3 days, even more preferably over about 3 days. For example, the 0.5 mg twice daily (BID) dose may be initially administered at 0.25 mg (or any other interim dose level) twice daily (BID) for three days (or any suitable period) before increasing the dosage to the final desired 0.5 mg twice daily (BID) dose; the 1 mg twice daily (BID) dose may be initially administered at 0.5 mg twice daily (or any other interim dose level) (BID) for three days (or any suitable period) before increasing the dosage to the final desired 1 mg twice daily (BID) dose; and the 1 mg once daily (QD) dose may be initially administered at 0.5 mg once daily (or any other interim dose level) (QD) for three days (or any suitable period) before increasing the dosage to the final desired 1 mg once daily (QD) dose.

In preferred embodiments, the compound of formula (IA) is a trans 3,4-isomer.

In certain embodiments employing compounds of formula (IA), it is preferred that
R¹ is hydrogen;
R² is alkyl;
n is 1 or 2;
R³ is benzyl, phenyl, cyclohexyl, or cyclohexylmethyl; and
R⁴ is alkyl.

In certain embodiments employing compounds of formula (IA), it is preferred that
A is OR⁵; and
R⁵ is hydrogen or alkyl.

In certain embodiments employing compounds of formula (IA), it is preferred that
A is NR⁶R⁷;
R⁶ is hydrogen;
R⁷ is alkylene substituted B; and
B is C(O)W.

In certain embodiments employing compounds of formula (IA), it is preferred that
R⁷ is (CH₂)_{q}—B;
q is about 1 to about 3;
W is OR¹⁰; and
R¹⁰ is hydrogen, alkyl, phenyl-substituted alkyl, cycloalkyl or cycloalkyl-substituted alkyl.

In certain embodiments including compounds of formula (IA), it is preferred that
W is NR¹¹R¹²
R¹¹ is hydrogen or alkyl; and
R¹² is hydrogen, alkyl or alkylene substituted C(═O)Y.

In certain embodiments employing compounds of formula (IA), it is preferred that
R¹² is (CH₂)ₘC(O)Y;
m is 1 to 3;
Y is OR¹⁸ or NR¹⁹R²⁰; and
R¹⁸, R¹⁹ and R²⁰ are independently hydrogen or alkyl.

In certain embodiments employing compounds of formula (IA), it is preferred that
W is OE;
E is CH₂C(═O)D;
D is OR¹⁵ or NR¹⁶R¹⁷;
R¹⁵ is hydrogen or alkyl;
R¹⁶ is methyl or benzyl; and
R¹⁷ is hydrogen.

In certain embodiments employing compounds of formula (IA), it is preferred that
W is OE;
E is R¹³OC(═O)R¹⁴;
R¹³ is —CH(CH₃)— or —CH(CH₂CH₃)—; and
R¹⁴ is alkyl.

In certain embodiments including compounds of formula (IA), it is preferred that p is 1.

In certain embodiments employing compounds of formula (IA), it is preferred that the configuration at positions 3 and 4 of the piperidine ring is each R.

Preferred compounds of formula (IA) include:
Q-CH₂CH(CH₂(C₆H₅))C(O)OH,
Q-CH₂CH₂CH(C₆H₅)C(O)NHCH₂C(O)OCH₂CH₃,
Q-CH₂CH₂CH(C₆H₅)C(O)NHCH₂C(O)OH,
Q-CH₂CH₂CH(C₆H₅)C(O)NHCH₂C(O)NHCH₃,
Q-CH₂CH₂CH(C₆H₅)C(O)NHCH₂C(O)NHCH₂CH₃,
G-NH(CH₂)₂C(O)NH₂,
G-NH(CH₂)₂C(O)NHCH₃,
G-NHCH₂C(O)NH₂,
G-NHCH₂C(O)NHCH₃,
G-NHCH₂C(O)NHCH₂CH₃,
G-NH(CH₂)₃C(O)OCH₂CH₃,
G-NH(CH₂)₃C(O)NHCH₃,
G-NH(CH₂)₂C(O)OH,
G-NH(CH₂)₃C(O)OH,
Q-CH₂CH(CH₂(C₆H₁₁))C(O)NH(CH₂)₂C(O)OH,
Q-CH₂CH(CH₂(C₆H₁₁))C(O)NH(CH₂)₂C(O)NH₂,
Z-NHCH₂C(O)OH,
Z-NHCH₂C(O)NH₂,
Z-NHCH₂C(O)N(CH₃)₂,
Z-NHCH₂C(O)NHCH(CH₃)₂,
Z-NH(CH₂)₂C(O)OCH₂(C₆H₅),
Z-NH(CH₂)₂C(O)NHCH₂CH₃,
Z-NH(CH₂)₃C(O)NHCH₃,
Z-NHCH₂C(O)NHCH₂C(O)OH,
Z-NHCH₂C(O)OCH₂C(O)OCH₃,
Z-NHCH₂C(O)OCH₂C(O)NHCH₃,
Z-NHCH₂C(O)O-(4-methoxycyclohexyl),
Z-NHCH₂C(O)OCH₂C(O)NHCH₂(C₆H₅) and
Z-NHCH₂C(O)OCH(CH₃)OC(O)CH₃;
wherein:

Q represents

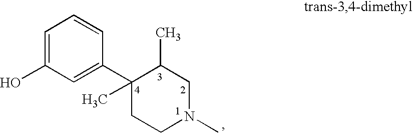

G represents

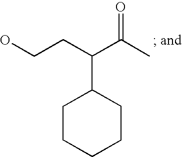

Z represents

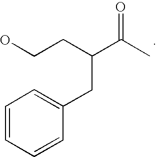

More preferred compounds of formula (IA) include:
(+)-Z-NHCH₂C(O)OH,
(−)-Z-NHCH₂C(O)OH,
(3R,4R)-Z-NHCH₂C(O)NHCH₂(C₆H₅) and
(3R,4R)-G-NH(CH₂)₃C(O)OH,

wherein Q, Z and G are as defined above.

Even more preferred compounds of formula (IA) include (+)-Z-NHCH₂C(O)OH and (−)-Z-NHCH₂C(O)OH, most especially (+)-Z-NHCH₂C(O)OH, where Z is as defined above.

Even more preferred compounds of formula (IA) include Q-CH₂CH(CH₂(C₆H₅))C(O)OH, wherein Q is as defined above. It is especially preferred when said compound is (3R,4R,S)-Q-CH₂CH(CH₂(C₆H₅))C(O)OH.

A particularly preferred embodiment of the present invention is the compound (+)-Z-NHCH₂C(O)OH, i.e., the compound of the following formula (II):

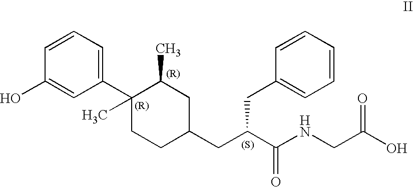

The compound of formula (II) has low solubility in water except at low or high pH conditions. Zwitterionic character may be inherent to the compound, and may impart desirable properties such as poor systemic absorption and sustained local affect on the gut following oral administration.

In especially preferred embodiments, the compound of a formula (IA) is a substantially pure stereoisomer.

The compounds employed in the methods of the present invention may exist in prodrug form. As used herein, “prodrug” is intended to include any covalently bonded carriers that release the active parent drug, for example, as according to formulas (IA) or other formulas or compounds employed in the methods of the present invention in vivo when such prodrug is administered to a mammalian subject. Since prodrugs are known to enhance numerous desirable qualities of pharmaceuticals (e.g., solubility, bioavailability, manufacturing, etc.) the compounds employed in the present methods may, if desired, be delivered in prodrug form. Thus, the present invention contemplates methods of delivering prodrugs. Prodrugs of the compounds employed in the present invention, for example formula (IA), may be prepared by modifying functional groups present in the compound in such a way that the modifications are cleaved, either in routine manipulation or in vivo, to the parent compound.

Accordingly, prodrugs include, for example, compounds described herein in which a hydroxy, amino, or carboxy group is bonded to any group that, when the prodrug is administered to a mammalian subject, cleaves to form a free hydroxyl, free amino, or carboxylic acid, respectively. Examples include, but are not limited to, acetate, formate and benzoate derivatives of alcohol and amine functional groups; and alkyl, carbocyclic, aryl, and alkylaryl esters such as methyl, ethyl, propyl, iso-propyl, butyl, isobutyl, sec-butyl, tert-butyl, cyclopropyl, phenyl, benzyl, and phenethyl esters, and the like.

In certain preferred embodiments, the 4-aryl-piperidine derivative or stereoisomer, prodrug, pharmaceutically acceptable salt, hydrate, solvate, acid salt hydrate, N-oxide or isomorphic form thereof is administered for at least about 2 weeks, preferably for at least about weeks.

In certain preferred embodiments, the patient experiences fewer, less, or fewer and less adverse events associated with the administration of 4-aryl piperidine derivative; wherein said adverse events are selected from the group consisting of nausea, abdominal pain, vomiting, diarrhea, flatulence, abdominal distension, and combinations thereof.

The compounds employed in the methods of the present invention may be prepared in a number of ways well known to those skilled in the art. The compounds can be synthesized, for example, using the methods described in U.S. Pat. No. 5,250,542, U.S. Pat. No. 6,469,030, and U.S. Pat. No. 6,451,806, the disclosures of which are hereby incorporated by reference, in their entireties. All processes disclosed in association with the present invention are contemplated to be practiced on any scale, including milligram, gram, multigram, kilogram, multikilogram or commercial industrial scale.

As discussed in detail above, compounds employed in the present methods may contain one or more asymmetrically substituted carbon atoms, and may be isolated in optically active or racemic forms. Thus, all chiral, diastereomeric, racemic forms and all geometric isomeric forms of a structure are intended, unless the specific stereochemistry or isomeric form is specifically indicated. It is well known in the art how to prepare and isolate such optically active forms. For example, mixtures of stereoisomers may be separated by standard techniques including, but not limited to, resolution of racemic forms, normal, reverse-phase, and chiral chromatography, preferential salt formation, recrystallization, and the like, or by chiral synthesis either from chiral starting materials or by deliberate synthesis of target chiral centers.

As will be readily understood, functional groups present may contain protecting groups during the course of synthesis. Protecting groups are known per se as chemical functional groups that can be selectively appended to and removed from functionalities, such as hydroxyl groups and carboxyl groups. These groups are present in a chemical compound to render such functionality inert to chemical reaction conditions to which the compound is exposed. Any of a variety of protecting groups may be employed with the present invention. Preferred protecting groups include the benzyloxycarbonyl group and the tert-butyloxycarbonyl group. Other preferred protecting groups that may be employed in accordance with the present invention may be described in Greene, T. W. and Wuts, P. G. M., Protective Groups in Organic Synthesis 2d. Ed., Wiley & Sons, 1991.

As noted above, the compounds of the present invention can exist as the individual stereoisomers. Preferably, reaction conditions are adjusted as disclosed in U.S. Pat. No. 4,581,456 or as set forth in Example 1 of U.S. Pat. No. 5,250,542 to be substantially stereoselective and provide a racemic mixture of essentially two enantiomers. These enantiomers may then be resolved. A procedure which may be employed to prepare the resolved starting materials used in the synthesis of these compounds includes treating a racemic mixture of alkyl-3,4-dimethyl-4-(3-alkoxyphenyl)piperidine with either (+)- or (−)-ditoluoyl tartaric acid to provide the resolved intermediate. This compound may then be dealkylated at the 1-position with vinyl chloroformate and finally converted to the desired 4-(3-hydroxyphenyl)piperidine isomer.

As will be understood by those skilled in the art, the individual enantiomers of the invention can also be isolated with either (+) or (−) dibenzoyl tartaric acid, as desired, from the corresponding racemic mixture of the compounds of the invention. Preferably, the (+)-trans enantiomer is obtained.

Although the (+)trans-3,4 stereoisomer is preferred, all of the possible stereoisomers of the compounds described herein are within the contemplated scope of the present invention. Racemic mixtures of the stereoisomers as well as the substantially pure stereoisomers are within the scope of the invention. The term “substantially pure,” as used herein, refers to at least about 90 mole percent, more preferably at least about 95 mole percent and most preferably at least about 98 mole percent of the desired stereoisomer is present relative to other possible stereoisomers.

Another synthetic route can involve the reaction of a substituted piperidine with a haloalkylnitrile. The nitrile group of the resulting piperidine alkylnitrile can be hydrolyzed to the corresponding carboxylic acid.

The compounds employed in the methods of the present invention may be administered by any means that results in the contact of the active agents with the agents' site or site(s) of action in the body of a patient. The compounds may be administered by any conventional means available for use in conjunction with pharmaceuticals, either as individual therapeutic agents or in a combination of therapeutic agents. For example, they may be administered as the sole active agents in a pharmaceutical composition, or they can be used in combination with other therapeutically active ingredients.

The compounds are preferably combined with a pharmaceutical carrier selected on the basis of the chosen route of administration and standard pharmaceutical practice as described, for example, in Remington's Pharmaceutical Sciences (Mack Publishing Co., Easton, Pa., 1980), the disclosures of which is hereby incorporated herein by reference, in its entirety.

Compounds of the present invention can be administered to a mammalian host in a variety of forms adapted to the chosen route of administration, e.g., orally. Other acceptable routes of administration are parenteral including intravenous; transepithelial including transdermal, transnasal, ophthalmic, sublingual and buccal; topical including ophthalmic, dermal, ocular, and rectal; nasal or pulmonary inhalation via insufflation or aerosol; and rectal systemic.

The active compound may be orally administered, for example, with an inert diluent or with an assimilable edible carrier, it may be enclosed in hard or soft shell gelatin capsules, it may be compressed into tablets, or it may be incorporated directly with the food of the diet. For oral therapeutic administration, the active compound may be incorporated with excipient and used in the form of ingestible tablets, buccal tablets, troches, capsules, elixirs, suspensions, syrups, wafers, and the like.

The active compound may also be administered parenterally. Solutions of the active compounds as free bases or pharmacologically acceptable salts can be prepared in water suitably mixed with a surfactant, such as hydroxypropylcellulose. A dispersion can also be prepared in glycerol, liquid polyethylene glycols and mixtures thereof and in oils. Under ordinary conditions of storage and use, these preparations may contain a preservative to prevent the growth of microorganisms.

The pharmaceutical forms suitable for injectable use include, for example, sterile aqueous solutions or dispersions and sterile powders for the extemporaneous preparation of sterile injectable solutions or dispersions. In all cases, the form is preferably sterile and fluid to provide easy syringability. It is preferably stable under the conditions of manufacture and storage and is preferably preserved against the contaminating action of microorganisms such as bacteria and fungi. The carrier may be a solvent or dispersion medium containing, for example, water, ethanol, polyol (for example, glycerol, propylene glycol, liquid polyethylene glycol and the like), suitable mixtures thereof, and vegetable oils. The proper fluidity can be maintained, for example, by the use of a coating, such as lecithin, by the maintenance of the required particle size in the case of a dispersion, and by the use of surfactants. The prevention of the action of microorganisms may be achieved by various antibacterial and antifungal agents, for example, parabens, chlorobutanol, phenol, sorbic acid, thimerosal and the like. In many cases, it will be preferable to include isotonic agents, for example, sugars or sodium chloride. Prolonged absorption of the injectable compositions may be achieved by the use of agents delaying absorption, for example, aluminum monostearate and gelatin.

Sterile injectable solutions may be prepared by incorporating the active compounds in the required amounts, in the appropriate solvent, with various of the other ingredients enumerated above, as required, followed by filtered sterilization. Generally, dispersions may be prepared by incorporating the sterilized active ingredient into a sterile vehicle which contains the basic dispersion medium and the required other ingredients from those enumerated above. In the case of sterile powders for the preparation of sterile injectable solutions, the preferred methods of preparation may include vacuum drying and the freeze-drying technique that yield a powder of the active ingredient, plus any additional desired ingredient from the previously sterile-filtered solution thereof.

The therapeutic compounds of this invention may be administered to a patient alone or in combination with a pharmaceutically acceptable carrier. As noted above, the relative proportions of active ingredient and carrier may be determined, for example, by the solubility and chemical nature of the compounds, chosen route of administration and standard pharmaceutical practice.

The dosage of the compounds of the present invention that will be most suitable for prophylaxis or treatment will vary with the form of administration, the particular compound chosen and the physiological characteristics of the particular patient under treatment. Generally, small dosages may be used initially and, if necessary, increased by small increments until the desired effect under the circumstances is reached. Generally speaking, oral administration may require higher dosages.

The combination products useful in the methods of this invention, such as pharmaceutical compositions comprising 4-aryl-piperidine derivatives with additional active ingredients, may be in any dosage form, such as those described herein, and can also be administered in various ways, as described herein. In a preferred embodiment, the combination products of the invention are formulated together, in a single dosage form (that is, combined together in one capsule, tablet, powder, or liquid, etc.). When the combination products are not formulated together in a single dosage form, the 4-aryl-piperidine derivative and additional active ingredient may be administered at the same time or simultaneously (that is, together), or in any order. When not administered at the same time or simultaneously, that is, when administered sequentially, preferably the administration of a 4-aryl-piperidine derivative and additional active ingredient occurs less than about one hour apart, more preferably less than about 30 minutes apart, even more preferably less than about 15 minutes apart, and still more preferably less than about 5 minutes apart.

Preferably, administration of the combination products of the invention is oral, although other routes of administration, as described above, are contemplated to be within the scope of the present invention. Although it is preferable that the 4-aryl-piperidine derivative and the additional active ingredients are all administered in the same fashion (that is, for example, both orally), if desired, they may each be administered in different fashions (that is, for example, one component of the combination product may be administered orally, and another component may be administered intravenously). The dosage of the combination products of the invention may vary depending upon various factors such as the pharmacodynamic characteristics of the particular agent and its mode and route of administration, the age, health and weight of the recipient, the nature and extent of the symptoms, the kind of concurrent treatment, the frequency of treatment, and the effect desired.

Particularly when provided as a single dosage form, the potential exists for a chemical interaction between the combined active ingredients. For this reason, the preferred dosage forms of the combination products of this invention are formulated such that although the active ingredients are combined in a single dosage form, the physical contact between the active ingredients is minimized (that is, reduced).

In order to minimize contact, one embodiment of this invention where the product is orally administered provides for a combination product wherein one active ingredient is enteric coated. By enteric coating one or more of the active ingredients, it is possible not only to minimize the contact between the combined active ingredients, but also, it is possible to control the release of one of these components in the gastrointestinal tract such that one of these components is not released in the stomach but rather is released in the intestines. Another embodiment of this invention where oral administration is desired provides for a combination product wherein one of the active ingredients is coated with a sustained-release material that effects a sustained-release throughout the gastrointestinal tract and also serves to minimize physical contact between the combined active ingredients. Furthermore, the sustained-released component can be additionally enteric coated such that the release of this component occurs only in the intestine. Still another approach would involve the formulation of a combination product in which the one component is coated with a sustained and/or enteric release polymer, and the other component is also coated with a polymer such as a low-viscosity grade of hydroxypropyl methylcellulose (HPMC) or other appropriate materials as known in the art, in order to further separate the active components. The polymer coating serves to form an additional barrier to interaction with the other component.

Dosage forms of the combination products of the present invention wherein one active ingredient is enteric coated can be in the form of tablets such that the enteric coated component and the other active ingredient are blended together and then compressed into a tablet or such that the enteric coated component is compressed into one tablet layer and the other active ingredient is compressed into an additional layer. Optionally, in order to further separate the two layers, one or more placebo layers may be present such that the placebo layer is between the layers of active ingredients. In addition, dosage forms of the present invention can be in the form of capsules wherein one active ingredient is compressed into a tablet or in the form of a plurality of microtablets, particles, granules or non-pareils, which are then enteric coated. These enteric coated microtablets, particles, granules or non-pareils are then placed into a capsule or compressed into a capsule along with a granulation of the other active ingredient.

These as well as other ways of minimizing contact between the components of combination products of the present invention, whether administered in a single dosage form or administered in separate forms but at the same time by the same manner, will be readily apparent to those skilled in the art, once armed with the present disclosure.

Pharmaceutical kits useful in the methods of the invention are also within the ambit of the present invention. Sterilization of the container may be carried out using conventional sterilization methodology well known to those skilled in the art. The sterile containers of materials may comprise separate containers, or one or more multi-part containers, as exemplified by the UNIVIAL™ two-part container (available from Abbott Labs, Chicago, Ill.), as desired. The 4-aryl-piperidine derivative and the optional additional active ingredient may be separate, or combined into a single dosage form as described above. Such kits may further include, if desired, one or more of various conventional pharmaceutical kit components, such as for example, one or more pharmaceutically acceptable carriers, additional vials for mixing the components, etc., as will be readily apparent to those skilled in the art. Instructions, either as inserts or as labels, indicating quantities of the components to be administered, guidelines for administration, and/or guidelines for mixing the components, may also be included in the kit.

### EXAMPLES

The present invention will now be illustrated by reference to the following specific, non-limiting examples. The examples are not intended to limit the scope of the present invention.

A six-week, randomize, double-blind, placebo-controlled study was conducted to evaluate three dosage regimens for [[(2S)-2-[[(3R,4R)-4-(3-hydroxyphenyl)-3,4-dimethylpiperidin-1-yl]methyl]-3-phenylpropanoyl]amino]acetic acid dihydrate (alvimopan) for the treatment of opioid-induced bowel dysfunction in non-malignant pain subjects, including an evaluation of the improvement of spontaneous bowel movement (SBM) frequency compared to placebo while maintaining an acceptable tolerability profile.

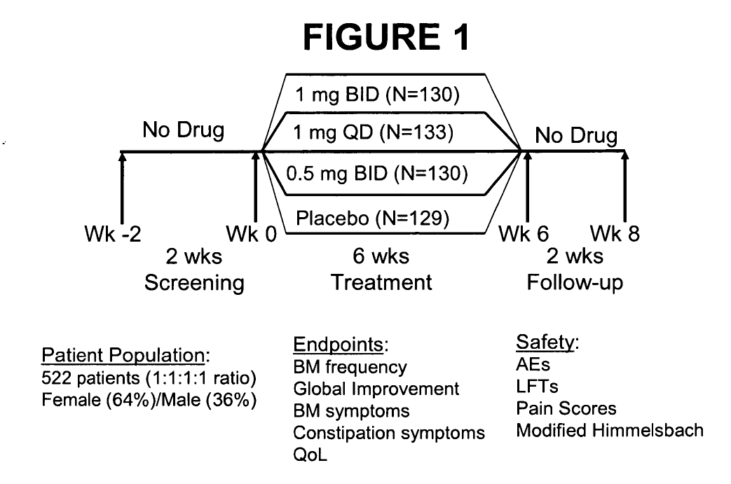

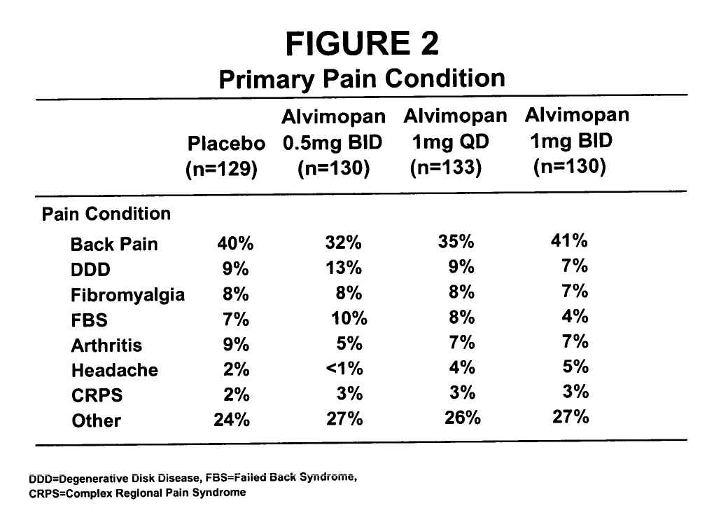

Subjects meeting the initial screening criteria for the study population, and who had been administered chronic opioid therapy for non-malignant pain, were selected. The study design as shown in FIG. 1 was carried out. The results are shown in FIGS. 2 to 22.

The primary pain condition for the subjects in each cohort is shown in FIG. 2.

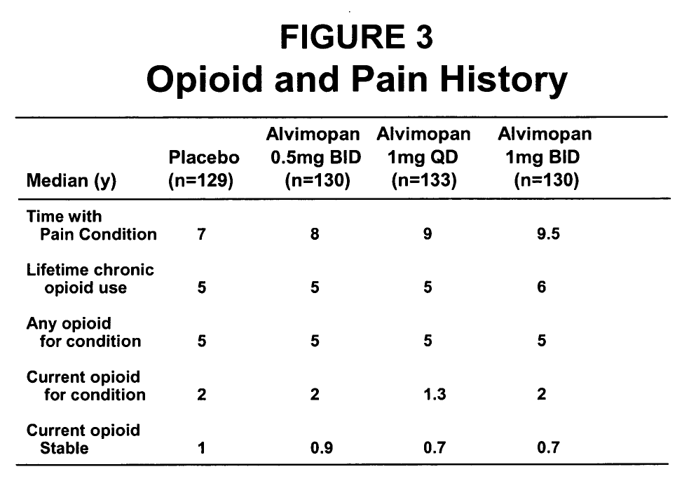

The opioid and pain history for the subjects in each cohort are shown in FIG. 3.

The opioid and pain history for the subjects in each cohort are shown in FIG. 3.

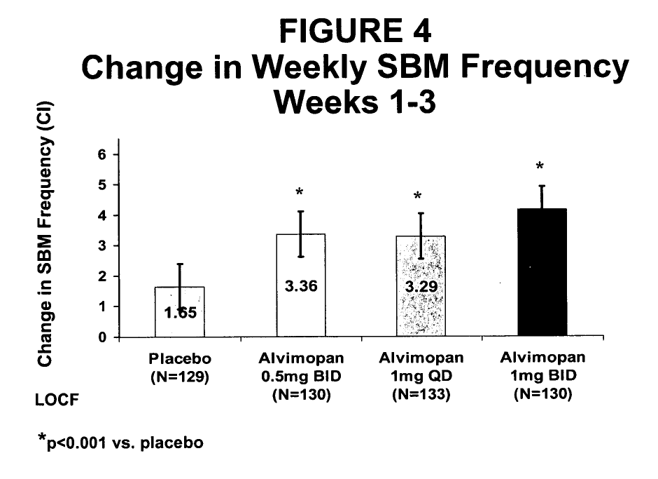

The change in weekly spontaneous bowel movements (SBM) frequency for weeks 1 to 3 is shown in FIG. 4.

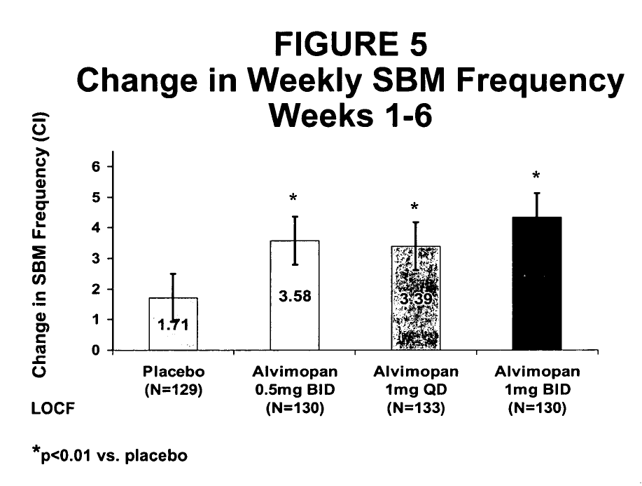

The change in weekly spontaneous bowel movements (SBM) frequency for weeks 1 to 6 is shown in FIG. 5.

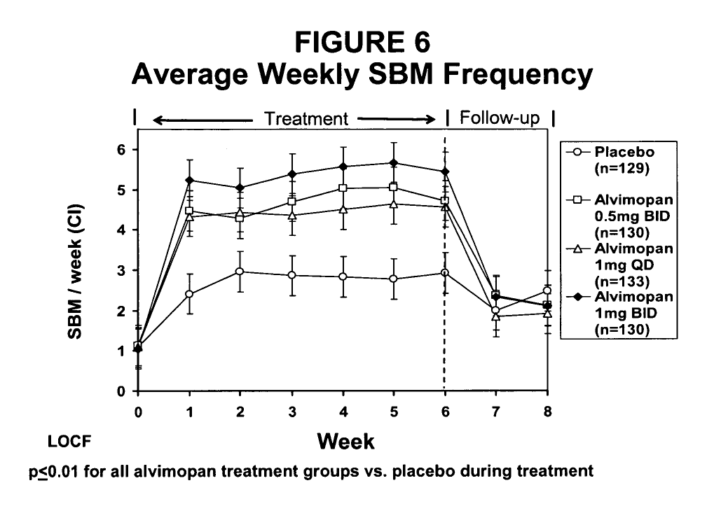

A plot of the average weekly SBM over time for the study is shown in FIG. 6.

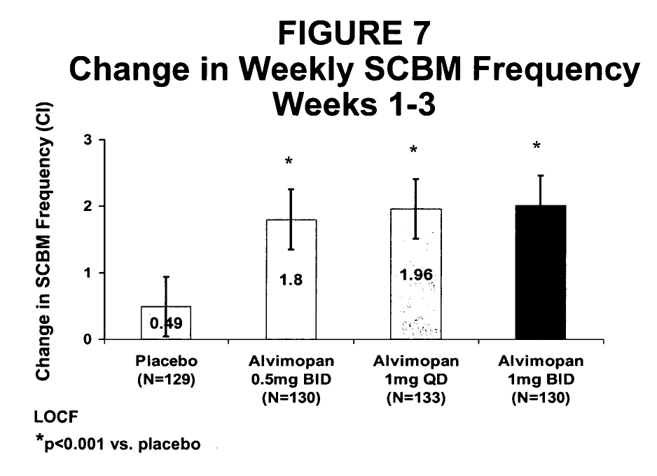

The change in weekly spontaneous complete bowel movements (SCBM) frequency for weeks 1 to 3 is shown in FIG. 7.

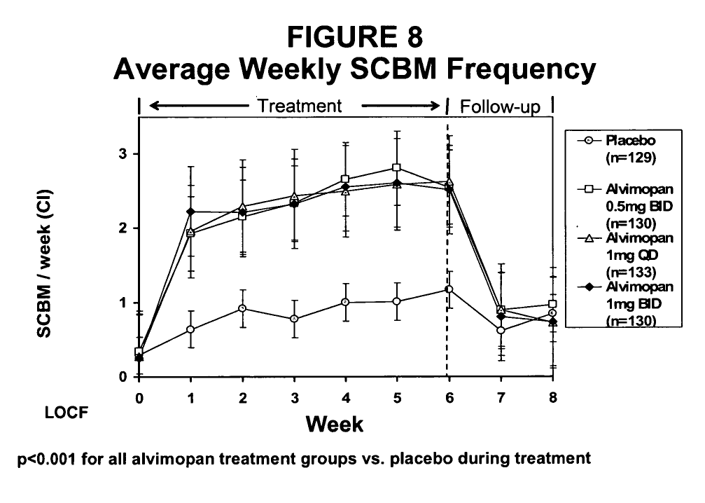

A plot of the average weekly SCBM over time for the study is shown in shown in FIG. 8.

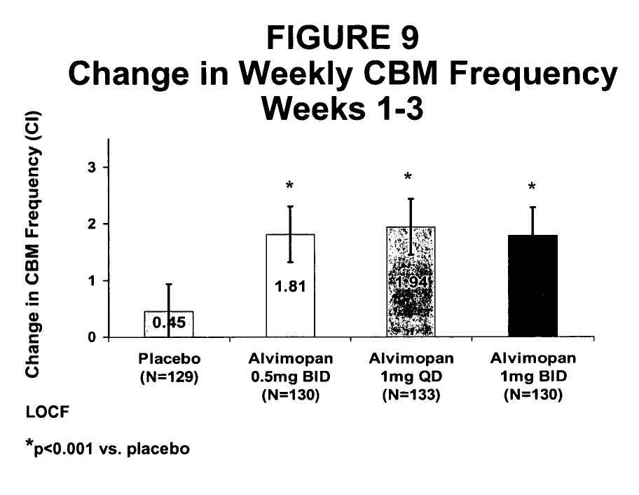

The change in weekly complete bowel movements (CBM) frequency for weeks 1 to 3 is shown in FIG. 9.

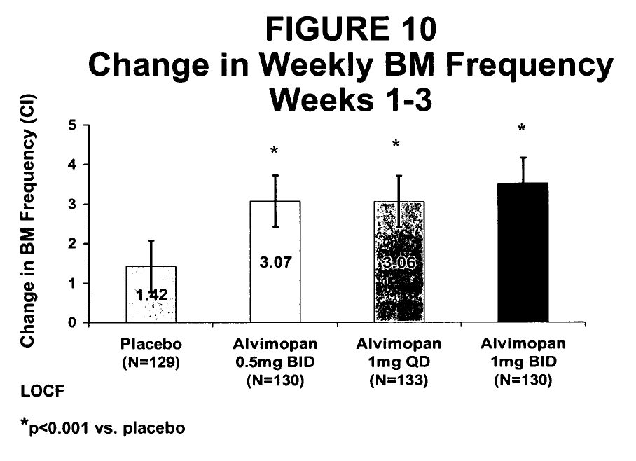

The change in weekly bowel movements (BM) frequency for weeks 1 to 3 is shown in FIG. 10.

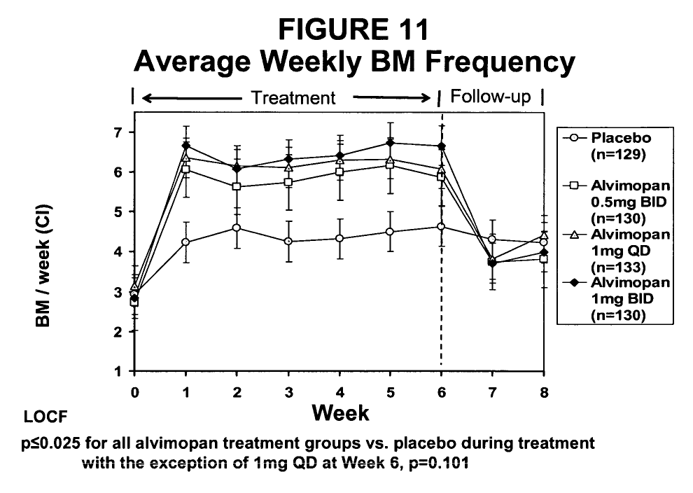

A plot of the average weekly BM over time for the study is shown in FIG. 11.

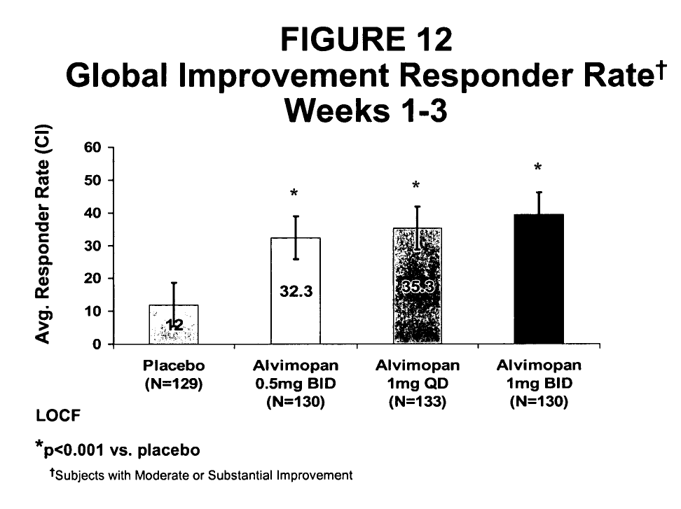

Global improvement responder rate for weeks 1 to 3 for the study is shown in FIG. 12.

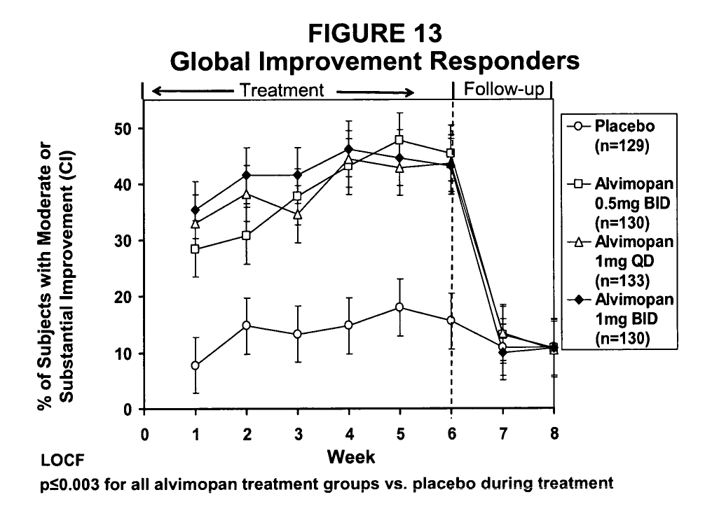

A plot of the global improvement responders over time for the study is shown in FIG. 13.

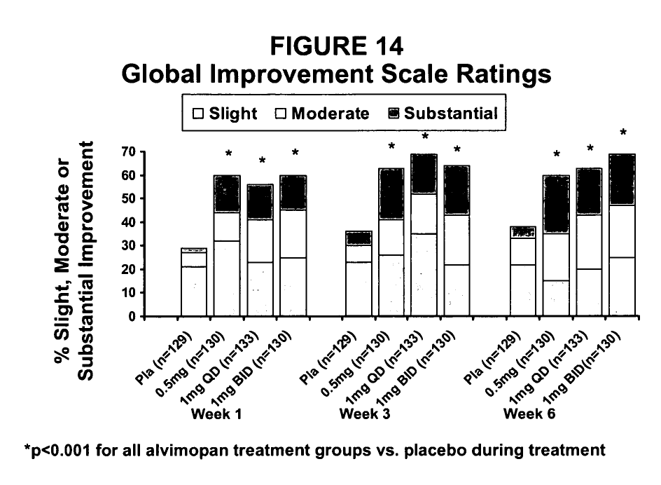

Global improvement scale ratings for the study are shown in FIG. 14.

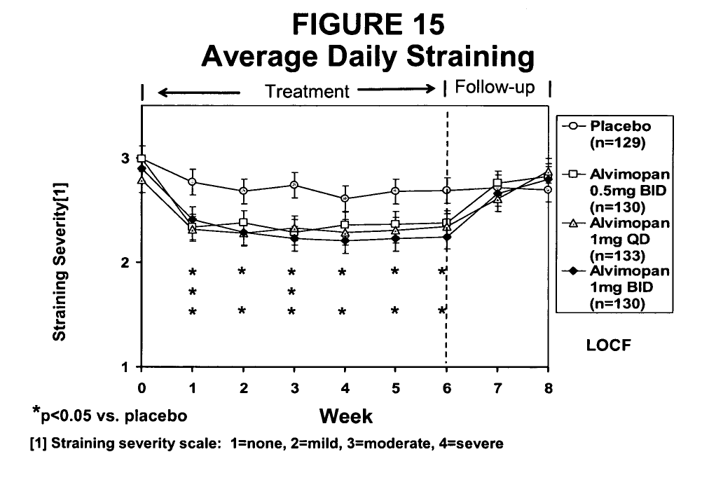

A plot of average daily straining over time for the study is shown in FIG. 15.

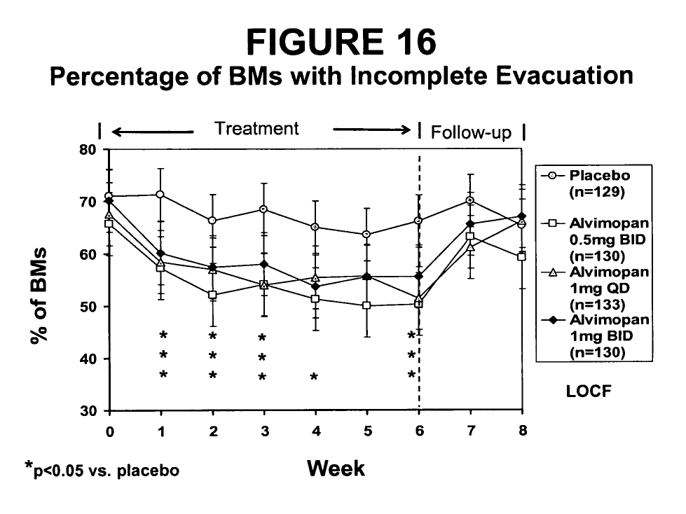

A plot of the percentage of BMs with incomplete evacuation for the study is shown in FIG. 16.

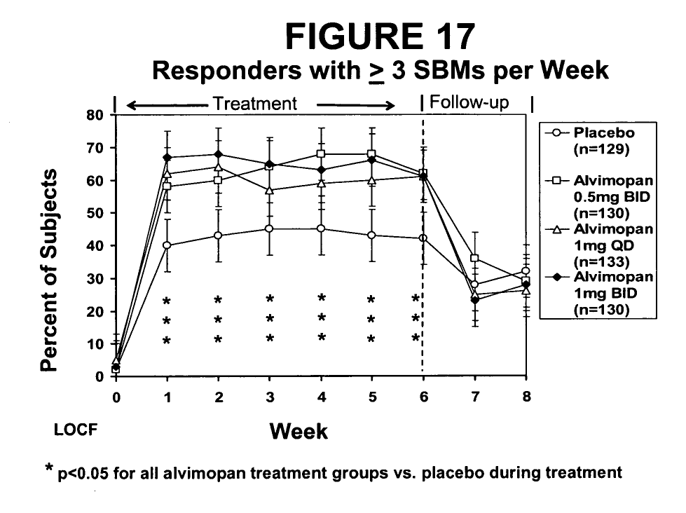

A plot of responders with at least 3 SBMs per week for the study is shown in FIG. 17.

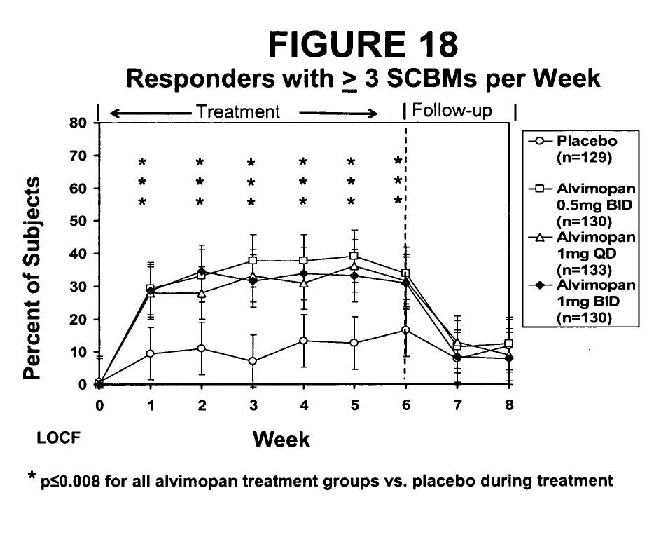

A plot of responders with at least 3 SCBMs per week for the study is shown in FIG. 18.

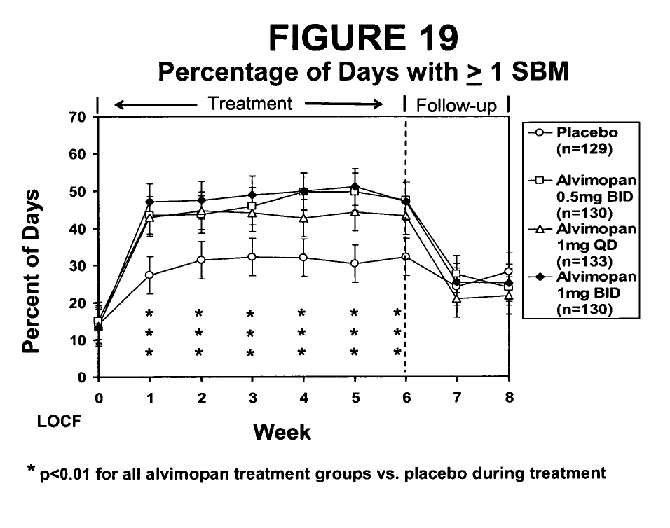

A plot of percentage of days with at least one SBM for the study is shown in FIG. 19.

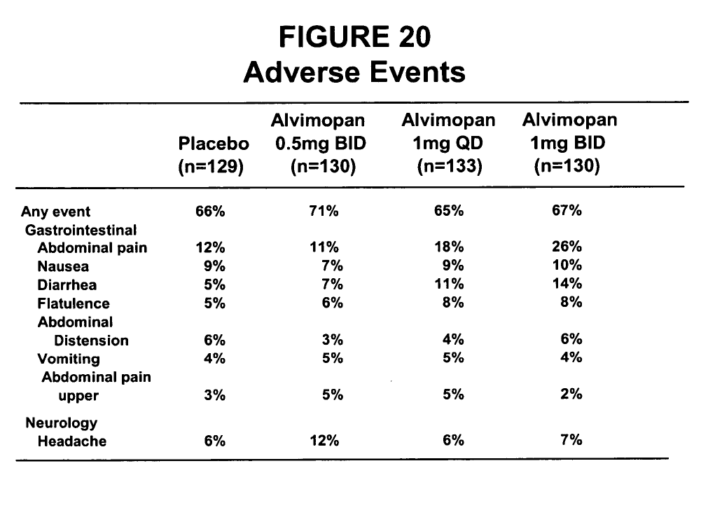

A chart of adverse events for each of the cohorts of the study is shown in FIG. 20.

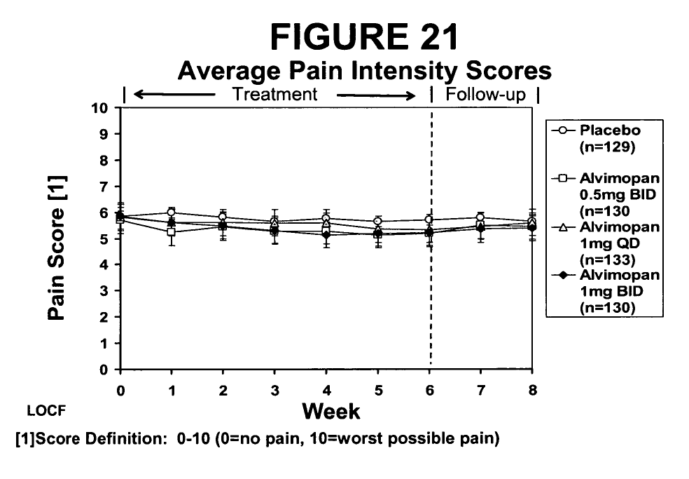

A plot of average pain intensity scores over time for the study is shown in FIG. 21.

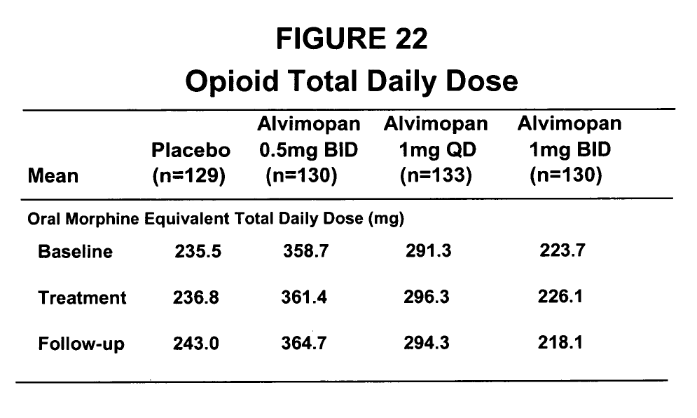

A chart of the opioid total daily dose for each of the cohorts of the study is shown in FIG. 22.

All three alvimopan doses were significantly better, both clinically and statistically, than the placebo for the primary key secondary endpoints. The treatment effects were significant at week 1 and were sustained over the entire six-week treatment period with no evidence of tachyphylaxis. In addition, there was a loss of treatment effect, rapid return of endpoints toward baseline during the non-treatment follow-up period with no evidence of rebound post active treatment.

In addition, there was no evidence of analgesic antagonism based on changes in pain scores or opioid consumption during the study. Also, all treatment arms were well-tolerated. Alvimopan (0.5 mg BID) demonstrated the most favorable benefit to risk profile with a safety profile comparable to the placebo with an efficacy comparable to 1 mg QD or 1 mg BID.

When ranges are used herein for physical properties, such as molecular weight, or chemical properties, such as chemical formulae, all combinations and subcombinations of ranges specific embodiments therein are intended to be included.

The disclosures of each patent, patent application, and publication cited or described in this document are hereby incorporated herein by reference, in their entirety.

Those skilled in the art will appreciate that numerous changes and modifications can be made to the preferred embodiments of the invention and that such changes and modifications can be made without departing from the spirit of the invention. It is, therefore, intended that the appended claims cover all such equivalent variations as fall within the true spirit and scope of the invention.

What is claimed is:

1. A method of treating or preventing opioid bowel dysfunction in a patient in need thereof, comprising the step of:
administering to said patient:
about 0.5 mg twice daily (BID);
about 1 mg once daily (QD); or
about 1 mg twice daily (BID);
of at least one 4-aryl-piperidine derivative or a stereoisomer, a prodrug, a pharmaceutically acceptable salt, a hydrate, a solvate, an acid salt hydrate, an N-oxide or an isomorphic form thereof;
wherein said patient is receiving chronic exogenous opioids for pain; and
wherein said 4-aryl-piperidine derivative is [[2-[[-4-(3-hydroxyphenyl)-3,4-dimethylpiperidin-1-yl]methyl]-3-phenylpropanoyl]amino]acetic acid.

2. A method of increasing the frequency of spontaneous complete bowel movements in a patient, comprising the step of:
administering to said patient a dosage level of:
about 0.5 mg twice daily (BID);
about 1 mg once daily (QD); or
about 1 mg twice daily (BID);
of at least one 4-aryl-piperidine derivative or a stereoisomer, a prodrug, a pharmaceutically acceptable salt, a hydrate, a solvate, an acid salt hydrate, an N-oxide or an isomorphic form thereof;
wherein said patient is receiving chronic exogenous opioids for pain; and
wherein said 4-aryl-piperidine derivative is [[2-[[-4-(3-hydroxyphenyl)-3,4-dimethylpiperidin-1-yl]methyl]-3-phenylpropanoyl]amino]acetic acid.

3. A method for improving the quality of life of a patient suffering from opioid bowel dysfunction, comprising the step of:
administering to said patient a dosage level of:
about 0.5 mg twice daily (BID);
about 1 mg once daily (QD); or
about 1 mg twice daily (BID);
of at least one 4-aryl-piperidine derivative or a stereoisomer, a prodrug, a pharmaceutically acceptable salt, a hydrate, a solvate, an acid salt hydrate, an N-oxide or an isomorphic form thereof;
wherein said patient is receiving chronic exogenous opioids for pain; and
wherein said 4-aryl-piperidine derivative is [[2-[[-4-(3-hydroxyphenyl)-3,4-dimethylpiperidin-1-yl]methyl]-3-phenylpropanoyl]amino]acetic acid.

4. A method for reducing a patient's dependence on laxatives, comprising the step of:
administering to said patient a dosage level of:
about 0.5 mg twice daily (BID);
about 1 mg once daily (QD); or
about 1 mg twice daily (BID);
of at least one 4-aryl-piperidine derivative or a stereoisomer, a prodrug, a pharmaceutically acceptable salt, a hydrate, a solvate, an acid salt hydrate, an N-oxide or an isomorphic form thereof;
wherein said patient is receiving chronic exogenous opioids for pain; and
wherein said 4-aryl-piperidine derivative is [[2-[[-4-(3-hydroxyphenyl)-3,4-dimethylpiperidin-1-yl]methyl]-3-phenylpropanoyl]amino]acetic acid.

5. A method for preventing or treating pain, comprising the step of:
administering to said patient at least one opioid; and
a dosage level of:
about 0.5 mg twice daily (BID);
about 1 mg once daily (QD); or
about 1 mg twice daily (BID);
of at least one 4-aryl-piperidine derivative or a stereoisomer, a prodrug, a pharmaceutically acceptable salt, a hydrate, a solvate, an acid salt hydrate, an N-oxide or an isomorphic form thereof;
wherein said 4-aryl-piperidine derivative is [[2-[[-4-(3-hydroxyphenyl)-3,4-dimethylpiperidin-1-yl]methyl]-3-phenylpropanoyl]amino]acetic acid.

6. A method according to claim 1,
wherein said [[2-[[-4-(3-hydroxyphenyl)-3,4-dimethylpiperidin-1-yl]methyl]-3-phenylpropanoyl]amino]acetic acid is in hydrate form.

7. A method according to claim 6,
wherein said [[2-[[-4-(3-hydroxyphenyl)-3,4-dimethylpiperidin-1-yl]methyl]-3-phenylpropanoyl]amino]acetic acid is [[2-[[-4-(3-hydroxyphenyl)-3,4-dimethylpiperidin-1-yl]methyl]-3-phenylpropanoyl]amino]acetic acid dihydrate.

8. A method according to claim 7,
wherein said compound is a substantially pure stereoisomer.

9. A method according to claim 8;
wherein said [[2-[[-4-(3-hydroxyphenyl)-3,4-dimethylpiperidin-1-yl]methyl]-3-phenylpropanoyl]amino]acetic acid is [[(2S)-2-[[(3R,4R)-4-(3-hydroxyphenyl)-3,4-dimethylpiperidin-1-yl]methyl]-3-phenylpropanoyl]amino]acetic acid dihydrate.

10. A method according to claim 1,
wherein said 4-aryl-piperidine derivative or stereoisomer, prodrug, pharmaceutically acceptable salt, hydrate, solvate, acid salt hydrate, N-oxide or isomorphic form thereof is administered for at least about 1 week to about 6 weeks.

11. A method according to claim 10,
wherein said 4-aryl-piperidine derivative or stereoisomer, prodrug, pharmaceutically acceptable salt, hydrate, solvate, acid salt hydrate, N-oxide or isomorphic form thereof is administered for at least about 3 weeks.

12. A method according to claim 1,
wherein said patient experiences fewer, less, or fewer and less adverse events;
wherein said adverse events are selected from the group consisting of nausea, abdominal pain, vomiting, diarrhea, and combinations thereof.

13. A method of treating or preventing opioid bowel dysfunction in a patient in need thereof, comprising the step of:
administering to said patient a dosage level of:
about 0.5 mg twice daily (BID);
about 1 mg once daily (QD); or
about 1 mg twice daily (BID);
of at least one 4-aryl-piperidine derivative or a stereoisomer, a prodrug, a pharmaceutically acceptable salt, a hydrate, a solvate, an acid salt hydrate, an N-oxide or an isomorphic form thereof;
wherein said patient is receiving chronic exogenous opioids for pain; and
wherein said 4-aryl-piperidine derivative is a compound of formula (IA): 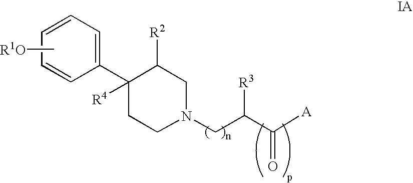
wherein:
R¹ is hydrogen or alkyl;
R² is hydrogen, alkyl or alkenyl;
R³ is hydrogen, alkyl, alkenyl, aryl, cycloalkyl, cycloalkenyl, cycloalkyl-substituted alkyl, cycloalkenyl-substituted alkyl or aralkyl;
R⁴ is hydrogen, alkyl or alkenyl;
A is OR⁵ or NR⁶R⁷;
R⁵ is hydrogen, alkyl, alkenyl, cycloalkyl, cycloalkenyl, cycloalkyl-substituted alkyl, cycloalkenyl-substituted alkyl, or aralkyl;
R⁶ is hydrogen or alkyl;
R⁷ is hydrogen, alkyl, alkenyl, cycloalkyl, aryl, cycloalkyl-substituted alkyl, cycloalkenyl, cycloalkenyl-substituted alkyl, aralkyl, B, or alkylene substituted B or, together with the nitrogen atom to which they are attached, R⁶ and R⁷ form a heterocyclic ring;
B is 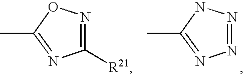
C(═O)W or NR⁸R⁹;
R⁸ is hydrogen or alkyl;
R⁹ is hydrogen, alkyl, alkenyl, cycloalkyl-substituted alkyl, cycloalkyl, cycloalkenyl, cycloalkenyl-substituted alkyl, aryl or aralkyl or, together with the nitrogen atom to which they are attached, R⁸ and R⁹ form a heterocyclic ring;
W is OR¹⁰, NR¹¹R¹², or OE;
R¹⁰ is hydrogen, alkyl, alkenyl, cycloalkyl, cycloalkenyl, cycloalkyl-substituted alkyl, cycloalkenyl-substituted alkyl, or aralkyl;
R¹¹ is hydrogen or alkyl;
R¹² is hydrogen, alkyl, alkenyl, aryl, cycloalkyl, cycloalkenyl, cycloalkyl-substituted alkyl, cycloalkenyl-substituted alkyl, aralkyl or alkylene substituted C(═O)Y or, together with the nitrogen atom to which they are attached, R¹¹ and R¹² form a heterocyclic ring;
E is 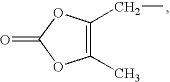
alkylene substituted (C═O)D, or —R¹³OC(═O)R¹⁴;
R¹³ is alkyl substituted alkylene;
R¹⁴ is alkyl;
D is OR¹⁵ or NR¹⁶R¹⁷;
R¹⁵ is hydrogen, alkyl, alkenyl, cycloalkyl, cycloalkenyl, cycloalkyl-substituted alkyl, cycloalkenyl-substituted alkyl, or aralkyl;
R¹⁶ is hydrogen, alkyl, alkenyl, aryl, aralkyl, cycloalkyl, cycloalkenyl, cycloalkyl-substituted alkyl or cycloalkenyl-substituted alkyl;
R¹⁷ is hydrogen or alkyl or, together with the nitrogen atom to which they are attached, R¹⁶ and R¹⁷ form a heterocyclic ring;
Y is OR¹⁸ or NR¹⁹R²⁰;
R¹⁸ is hydrogen, alkyl, alkenyl, cycloalkyl, cycloalkenyl, cycloalkyl-substituted alkyl, cycloalkenyl-substituted alkyl, or aralkyl;
R¹⁹ is hydrogen or alkyl;
R²⁰ is hydrogen, alkyl, alkenyl, aryl, cycloalkyl, cycloalkenyl, cycloalkyl-substituted alkyl, cycloalkenyl-substituted alkyl, or aralkyl or, together with the nitrogen atom to which they are attached, R¹⁹ and R²⁰ form a heterocyclic ring;
R²¹ is hydrogen or alkyl;
n is 0 to 4;
p is 0 or 1; and
provided that R¹⁰ is not hydrogen, when R¹ is hydrogen, R² is methyl, R³ is cycloalkyl-substituted alkyl, and R⁴ is methyl; and
provided that R¹⁰ is not alkyl, when R¹ is hydrogen, R² is methyl, R³ is aralkyl, and R⁴ is methyl.

14. A method of increasing the frequency of spontaneous complete bowel movements in a patient, comprising the step of:
administering to said patient a dosage level of:
about 0.5 mg twice daily (BID);
about 1 mg once daily (QD); or
about 1 mg twice daily (BID);
of at least one 4-aryl-piperidine derivative or a stereoisomer, a prodrug, a pharmaceutically acceptable salt, a hydrate, a solvate, an acid salt hydrate, an N-oxide or an isomorphic form thereof;
wherein said patient is receiving chronic exogenous opioids for pain; and
wherein said 4-aryl-piperidine derivative is a compound of formula (IA): 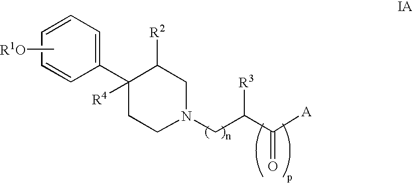
wherein:
R¹ is hydrogen or alkyl;
R² is hydrogen, alkyl or alkenyl;
R³ is hydrogen, alkyl, alkenyl, aryl, cycloalkyl, cycloalkenyl, cycloalkyl-substituted alkyl, cycloalkenyl-substituted alkyl or aralkyl;
R⁴ is hydrogen, alkyl or alkenyl;
A is OR⁵ or NR⁶R⁷;
R⁵ is hydrogen, alkyl, alkenyl, cycloalkyl, cycloalkenyl, cycloalkyl-substituted alkyl, cycloalkenyl-substituted alkyl, or aralkyl;
R⁶ is hydrogen or alkyl;
R⁷ is hydrogen, alkyl, alkenyl, cycloalkyl, aryl, cycloalkyl-substituted alkyl, cycloalkenyl, cycloalkenyl-substituted alkyl, aralkyl, B, or alkylene substituted B or, together with the nitrogen atom to which they are attached, R⁶ and R⁷ form a heterocyclic ring;
B is 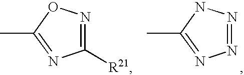
C(═O)W or NR⁸R⁹;
R⁸ is hydrogen or alkyl;
R⁹ is hydrogen, alkyl, alkenyl, cycloalkyl-substituted alkyl, cycloalkyl, cycloalkenyl, cycloalkenyl-substituted alkyl, aryl or aralkyl or, together with the nitrogen atom to which they are attached, R⁸ and R⁹ form a heterocyclic ring;
W is OR¹⁰, NR¹¹R¹², or OE;
R¹⁰ is hydrogen, alkyl, alkenyl, cycloalkyl, cycloalkenyl, cycloalkyl-substituted alkyl, cycloalkenyl-substituted alkyl, or aralkyl;
R¹¹ is hydrogen or alkyl;
R¹² is hydrogen, alkyl, alkenyl, aryl, cycloalkyl, cycloalkenyl, cycloalkyl-substituted alkyl, cycloalkenyl-substituted alkyl, aralkyl or alkylene substituted C(═O)Y or, together with the nitrogen atom to which they are attached, R¹¹ and R¹² form a heterocyclic ring;
E is 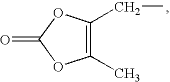
alkylene substituted (C═O)D, or —R¹³OC(═O)R¹⁴;
R¹³ is alkyl substituted alkylene;
R¹⁴ is alkyl;
D is OR¹⁵ or NR¹⁶R¹⁷;
R¹⁵ is hydrogen, alkyl, alkenyl, cycloalkyl, cycloalkenyl, cycloalkyl-substituted alkyl, cycloalkenyl-substituted alkyl, or aralkyl;
R¹⁶ is hydrogen, alkyl, alkenyl, aryl, aralkyl, cycloalkyl, cycloalkenyl, cycloalkyl-substituted alkyl or cycloalkenyl-substituted alkyl;
R¹⁷ is hydrogen or alkyl or, together with the nitrogen atom to which they are attached, R¹⁶ and R¹⁷ form a heterocyclic ring;
Y is OR¹⁸ or NR¹⁹R²⁰;
R¹⁸ is hydrogen, alkyl, alkenyl, cycloalkyl, cycloalkenyl, cycloalkyl-substituted alkyl, cycloalkenyl-substituted alkyl, or aralkyl;
R¹⁹ is hydrogen or alkyl;
R²⁰ is hydrogen, alkyl, alkenyl, aryl, cycloalkyl, cycloalkenyl, cycloalkyl-substituted alkyl, cycloalkenyl-substituted alkyl, or aralkyl or, together with the nitrogen atom to which they are attached, R¹⁹ and R²⁰ form a heterocyclic ring;
R²¹ is hydrogen or alkyl;
n is 0 to 4;
p is 0 or 1; and
provided that R¹⁰ is not hydrogen, when R¹ is hydrogen, R² is methyl, R³ is cycloalkyl-substituted alkyl, and R⁴ is methyl; and
provided that R¹⁰ is not alkyl, when R¹ is hydrogen, R² is methyl, R³ is aralkyl, and R⁴ is methyl.

15. A method for improving the quality of life of a patient suffering from opioid bowel dysfunction, comprising the step of:
administering to said patient a dosage level of:
about 0.5 mg twice daily (BID);
about 1 mg once daily (QD); or
about 1 mg twice daily (BID);
of at least one 4-aryl-piperidine derivative or a stereoisomer, a prodrug, a pharmaceutically acceptable salt, a hydrate, a solvate, an acid salt hydrate, an N-oxide or an isomorphic form thereof;
wherein said patient is receiving chronic exogenous opioids for pain; and
wherein said 4-aryl-piperidine derivative is a compound of formula (IA): 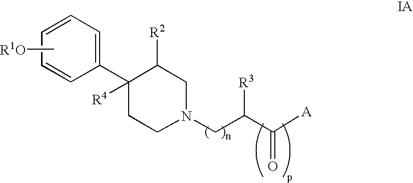
wherein:
R¹ is hydrogen or alkyl;
R² is hydrogen, alkyl or alkenyl;
R³ is hydrogen, alkyl, alkenyl, aryl, cycloalkyl, cycloalkenyl, cycloalkyl-substituted alkyl, cycloalkenyl-substituted alkyl or aralkyl;
R⁴ is hydrogen, alkyl or alkenyl;
A is OR⁵ or NR⁶R⁷;
R⁵ is hydrogen, alkyl, alkenyl, cycloalkyl, cycloalkenyl, cycloalkyl-substituted alkyl, cycloalkenyl-substituted alkyl, or aralkyl;
R⁶ is hydrogen or alkyl;
R⁷ is hydrogen, alkyl, alkenyl, cycloalkyl, aryl, cycloalkyl-substituted alkyl, cycloalkenyl, cycloalkenyl-substituted alkyl, aralkyl, B, or alkylene substituted B or, together with the nitrogen atom to which they are attached, R⁶ and R⁷ form a heterocyclic ring;
B is 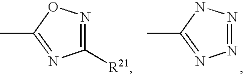
C(═O)W or NR⁸R⁹;
R⁸ is hydrogen or alkyl;
R⁹ is hydrogen, alkyl, alkenyl, cycloalkyl-substituted alkyl, cycloalkyl, cycloalkenyl, cycloalkenyl-substituted alkyl, aryl or aralkyl or, together with the nitrogen atom to which they are attached, R⁸ and R⁹ form a heterocyclic ring;
W is OR¹⁰, NR¹¹R¹², or OE;
R¹⁰ is hydrogen, alkyl, alkenyl, cycloalkyl, cycloalkenyl, cycloalkyl-substituted alkyl, cycloalkenyl-substituted alkyl, or aralkyl;
R¹¹ is hydrogen or alkyl;
R¹² is hydrogen, alkyl, alkenyl, aryl, cycloalkyl, cycloalkenyl, cycloalkyl-substituted alkyl, cycloalkenyl-substituted alkyl, aralkyl or alkylene substituted C(═O)Y or, together with the nitrogen atom to which they are attached, R¹¹ and R¹² form a heterocyclic ring;
E is 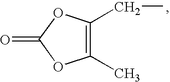
alkylene substituted (C═O)D, or —R¹³OC(═O)R¹⁴;
R¹³ is alkyl substituted alkylene;
R¹⁴ is alkyl;
D is OR¹⁵ or NR¹⁶R¹⁷;
R¹⁵ is hydrogen, alkyl, alkenyl, cycloalkyl, cycloalkenyl, cycloalkyl-substituted alkyl, cycloalkenyl-substituted alkyl, or aralkyl;
R¹⁶ is hydrogen, alkyl, alkenyl, aryl, aralkyl, cycloalkyl, cycloalkenyl, cycloalkyl-substituted alkyl or cycloalkenyl-substituted alkyl;
R¹⁷ is hydrogen or alkyl or, together with the nitrogen atom to which they are attached, R¹⁶ and R¹⁷ form a heterocyclic ring;
Y is OR¹⁸ or NR¹⁹R²⁰;
R¹⁸ is hydrogen, alkyl, alkenyl, cycloalkyl, cycloalkenyl, cycloalkyl-substituted alkyl, cycloalkenyl-substituted alkyl, or aralkyl;
R¹⁹ is hydrogen or alkyl;
R²⁰ is hydrogen, alkyl, alkenyl, aryl, cycloalkyl, cycloalkenyl, cycloalkyl-substituted alkyl, cycloalkenyl-substituted alkyl, or aralkyl or, together with the nitrogen atom to which they are attached, R¹⁹ and R²⁰ form a heterocyclic ring;
R²¹ is hydrogen or alkyl;
n is 0 to 4;
p is 0 or 1; and
provided that R¹⁰ is not hydrogen, when R¹ is hydrogen, R² is methyl, R³ is cycloalkyl-substituted alkyl, and R⁴ is methyl; and
provided that R¹⁰ is not alkyl, when R¹ is hydrogen, R² is methyl, R³ is aralkyl, and R⁴ is methyl.

16. A method for reducing a patient's dependence on laxatives, comprising the step of:
administering to said patient a dosage level of:
about 0.5 mg twice daily (BID);
about 1 mg once daily (QD); or
about 1 mg twice daily (BID);
of at least one 4-aryl-piperidine derivative or a stereoisomer, a prodrug, a pharmaceutically acceptable salt, a hydrate, a solvate, an acid salt hydrate, an N-oxide or an isomorphic form thereof;
wherein said patient is receiving chronic exogenous opioids for pain; and
wherein said 4-aryl-piperidine derivative is a compound of formula (IA): 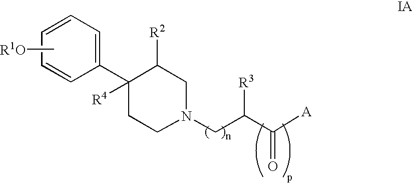
wherein:
R¹ is hydrogen or alkyl;
R² is hydrogen, alkyl or alkenyl;
R³ is hydrogen, alkyl, alkenyl, aryl, cycloalkyl, cycloalkenyl, cycloalkyl-substituted alkyl, cycloalkenyl-substituted alkyl or aralkyl;
R⁴ is hydrogen, alkyl or alkenyl;
A is OR⁵ or NR⁶R⁷;
R⁵ is hydrogen, alkyl, alkenyl, cycloalkyl, cycloalkenyl, cycloalkyl-substituted alkyl, cycloalkenyl-substituted alkyl, or aralkyl;
R⁶ is hydrogen or alkyl;
R⁷ is hydrogen, alkyl, alkenyl, cycloalkyl, aryl, cycloalkyl-substituted alkyl, cycloalkenyl, cycloalkenyl-substituted alkyl, aralkyl, B, or alkylene substituted B or, together with the nitrogen atom to which they are attached, R⁶ and R⁷ form a heterocyclic ring;
B is 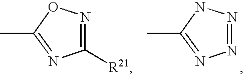
C(═O)W or NR⁸R⁹;
R⁸ is hydrogen or alkyl;
R⁹ is hydrogen, alkyl, alkenyl, cycloalkyl-substituted alkyl, cycloalkyl, cycloalkenyl, cycloalkenyl-substituted alkyl, aryl or aralkyl or, together with the nitrogen atom to which they are attached, R⁸ and R⁹ form a heterocyclic ring;
W is OR¹⁰, NR¹¹R¹², or OE;
R¹⁰ is hydrogen, alkyl, alkenyl, cycloalkyl, cycloalkenyl, cycloalkyl-substituted alkyl, cycloalkenyl-substituted alkyl, or aralkyl;
R¹¹ is hydrogen or alkyl;
R¹² is hydrogen, alkyl, alkenyl, aryl, cycloalkyl, cycloalkenyl, cycloalkyl-substituted alkyl, cycloalkenyl-substituted alkyl, aralkyl or alkylene substituted C(═O)Y or, together with the nitrogen atom to which they are attached, R¹¹ and R¹² form a heterocyclic ring;
E is 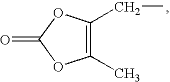
alkylene substituted (C═O)D, or —R¹³OC(═O)R¹⁴;
R¹³ is alkyl substituted alkylene;
R¹⁴ is alkyl;
D is OR¹⁵ or NR¹⁶R¹⁷;
R¹⁵ is hydrogen, alkyl, alkenyl, cycloalkyl, cycloalkenyl, cycloalkyl-substituted alkyl, cycloalkenyl-substituted alkyl, or aralkyl;
R¹⁶ is hydrogen, alkyl, alkenyl, aryl, aralkyl, cycloalkyl, cycloalkenyl, cycloalkyl-substituted alkyl or cycloalkenyl-substituted alkyl;
R¹⁷ is hydrogen or alkyl or, together with the nitrogen atom to which they are attached, R¹⁶ and R¹⁷ form a heterocyclic ring;
Y is OR¹⁸ or NR¹⁹R²⁰;
R¹⁸ is hydrogen, alkyl, alkenyl, cycloalkyl, cycloalkenyl, cycloalkyl-substituted alkyl, cycloalkenyl-substituted alkyl, or aralkyl;
R¹⁹ is hydrogen or alkyl;
R²⁰ is hydrogen, alkyl, alkenyl, aryl, cycloalkyl, cycloalkenyl, cycloalkyl-substituted alkyl, cycloalkenyl-substituted alkyl, or aralkyl or, together with the nitrogen atom to which they are attached, R¹⁹ and R²⁰ form a heterocyclic ring;
R²¹ is hydrogen or alkyl;
n is 0 to 4;
p is 0 or 1; and
provided that R¹⁰ is not hydrogen, when R¹ is hydrogen, R² is methyl, R³ is cycloalkyl-substituted alkyl, and R⁴ is methyl; and
provided that R¹⁰ is not alkyl, when R¹ is hydrogen, R² is methyl, R³ is aralkyl, and R⁴ is methyl.

17. A method for preventing or treating pain, comprising the step of:
administering to said patient at least one opioid; and
a dosage level of:
about 0.5 mg twice daily (BID);
about 1 mg once daily (QD); or
about 1 mg twice daily (BID);
of at least one 4-aryl-piperidine derivative or a stereoisomer, a prodrug, a pharmaceutically acceptable salt, a hydrate, a solvate, an acid salt hydrate, an N-oxide or an isomorphic form thereof;
wherein said 4-aryl-piperidine derivative is a compound of formula (IA): 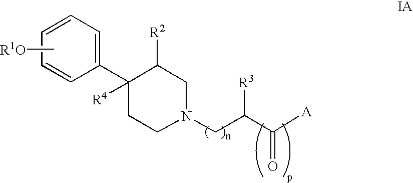
wherein:
R¹ is hydrogen or alkyl;
R² is hydrogen, alkyl or alkenyl;
R³ is hydrogen, alkyl, alkenyl, aryl, cycloalkyl, cycloalkenyl, cycloalkyl-substituted alkyl, cycloalkenyl-substituted alkyl or aralkyl;
R⁴ is hydrogen, alkyl or alkenyl;
A is OR⁵ or NR⁶R⁷;
R⁵ is hydrogen, alkyl, alkenyl, cycloalkyl, cycloalkenyl, cycloalkyl-substituted alkyl, cycloalkenyl-substituted alkyl, or aralkyl;
R⁶ is hydrogen or alkyl;
R⁷ is hydrogen, alkyl, alkenyl, cycloalkyl, aryl, cycloalkyl-substituted alkyl, together with the nitrogen atom to which they are attached, R⁶ and R⁷ form a heterocyclic ring;
B is 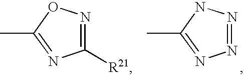
C(═O)W or NR⁸R⁹;
R⁸ is hydrogen or alkyl;
R⁹ is hydrogen, alkyl, alkenyl, cycloalkyl-substituted alkyl, cycloalkyl, cycloalkenyl, cycloalkenyl-substituted alkyl, aryl or aralkyl or, together with the nitrogen atom to which they are attached, R⁸ and R⁹ form a heterocyclic ring;
W is OR¹⁰, NR¹¹R¹², or OE;
R¹⁰ is hydrogen, alkyl, alkenyl, cycloalkyl, cycloalkenyl, cycloalkyl-substituted alkyl, cycloalkenyl-substituted alkyl, or aralkyl;
R¹¹ is hydrogen or alkyl;
R¹² is hydrogen, alkyl, alkenyl, aryl, cycloalkyl, cycloalkenyl, cycloalkyl-substituted alkyl, cycloalkenyl-substituted alkyl, aralkyl or alkylene substituted C(═O)Y or, together with the nitrogen atom to which they are attached, R¹¹ and R¹² form a heterocyclic ring;
E is 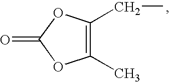
alkylene substituted (C═O)D, or —R¹³OC(═O)R¹⁴;
R¹³ is alkyl substituted alkylene;
R¹⁴ is alkyl;
D is OR¹⁵ or NR¹⁶R¹⁷;
R¹⁵ is hydrogen, alkyl, alkenyl, cycloalkyl, cycloalkenyl, cycloalkyl-substituted alkyl, cycloalkenyl-substituted alkyl, or aralkyl;
R¹⁶ is hydrogen, alkyl, alkenyl, aryl, aralkyl, cycloalkyl, cycloalkenyl, cycloalkyl-substituted alkyl or cycloalkenyl-substituted alkyl;
R¹⁷ is hydrogen or alkyl or, together with the nitrogen atom to which they are attached, R¹⁶ and R¹⁷ form a heterocyclic ring;
Y is OR¹⁸ or NR¹⁹R²⁰;
R¹⁸ is hydrogen, alkyl, alkenyl, cycloalkyl, cycloalkenyl, cycloalkyl-substituted alkyl, cycloalkenyl-substituted alkyl, or aralkyl;
R¹⁹ is hydrogen or alkyl;
R²⁰ is hydrogen, alkyl, alkenyl, aryl, cycloalkyl, cycloalkenyl, cycloalkyl-substituted alkyl, cycloalkenyl-substituted alkyl, or aralkyl or, together with the nitrogen atom to which they are attached, R¹⁹ and R²⁰ form a heterocyclic ring;
R²¹ is hydrogen or alkyl;
n is 0 to 4;
p is 0 or 1; and
provided that R¹⁰ is not hydrogen, when R¹ is hydrogen, R² is methyl, R³ is cycloalkyl-substituted alkyl, and R⁴ is methyl; and
provided that R¹⁰ is not alkyl, when R¹ is hydrogen, R² is methyl, R³ is aralkyl, and R⁴ is methyl.

18. A method according to claim 13,
wherein said 4-aryl-piperidine derivative or stereoisomer, prodrug, pharmaceutically acceptable salt, hydrate, solvate, acid salt hydrate, N-oxide or isomorphic form thereof is administered for at least about 1 week to about 6 weeks.

19. A method according to claim 18,
wherein said 4-aryl-piperidine derivative or stereoisomer, prodrug, pharmaceutically acceptable salt, hydrate, solvate, acid salt hydrate, N-oxide or isomorphic form thereof is administered for at least about 3 weeks.

20. A method according to claim 13,
wherein said patient experiences fewer, less, or fewer and less adverse events;
wherein said adverse events are selected from the group consisting of nausea, abdominal pain, vomiting, diarrhea, and combinations thereof.

21. A method according to claim 13,
wherein the compound of formula (IA) is a trans 3,4-isomer.

22. A method according to claim 13,
wherein:
R¹ is hydrogen;
R² is alkyl;
n is 1 or 2;
R³ is benzyl, phenyl, cyclohexyl, or cyclohexylmethyl; and
R⁴ is alkyl.

23. A method according to claim 13,
wherein:
A is OR⁵; and
R⁵ is hydrogen or alkyl.

24. A method according to claim 13,
wherein:
A is NR⁶R⁷;
R⁶ is hydrogen;
R⁷ is alkylene substituted B; and
B is C(O)W.

25. A method according to claim 13,
wherein:
R⁷ is (CH₂)_{q}—B;
q is about 1 to about 3;
W is OR¹⁰; and
R¹⁰ is hydrogen, alkyl, phenyl-substituted alkyl, cycloalkyl or cycloalkyl-substituted alkyl.

26. A method according to claim 13,
wherein:
W is NR¹¹R¹²
R¹¹ is hydrogen or alkyl; and
R¹² is hydrogen, alkyl or alkylene substituted C(═O)Y.

27. A method according to claim 13,
wherein:
R¹² is (CH₂)ₘC(O)Y;
m is 1 to 3;
Y is OR¹⁸ or NR¹⁹R²⁰; and
R¹⁸, R¹⁹ and R²⁰ are independently hydrogen or alkyl.

28. A method according to claim 13,
wherein:
W is OE;
E is CH₂C(═O)D;
D is OR¹⁵ or NR¹⁶R¹⁷;
R¹⁵ is hydrogen or alkyl;
R¹⁶ is methyl or benzyl; and
R¹⁷ is hydrogen.

29. A method according to claim 13,
wherein:
W is OE;
E is R¹³OC(═O)R¹⁴;
R¹³ is —CH(CH₃)— or —CH(CH₂CH₃)—; and
R¹⁴ is alkyl.

30. A method according to claim 13,
wherein p is 1.

31. A method according to claim 13,
wherein the configuration at positions 3 and 4 of the piperidine ring is each R.

32. A method according to claim 13,
wherein said compound is selected from the group consisting of:
Q-CH₂CH(CH₂(C₆H₅))C(O)OH,
Q-CH₂CH₂CH(C₆H₅)C(O)NHCH₂C(O)OCH₂CH₃,
Q-CH₂CH₂CH(C₆H₅)C(O)NHCH₂C(O)OH,
Q-CH₂CH₂CH(C₆H₅)C(O)NHCH₂C(O)NHCH₃,
Q-CH₂CH₂CH(C₆H₅)C(O)NHCH₂C(O)NHCH₂CH₃,
G-NH(CH₂)₂C(O)NH₂,
G-NH(CH₂)₂C(O)NHCH₃,
G-NHCH₂C(O)NH₂,
G-NHCH₂C(O)NHCH₃,
G-NHCH₂C(O)NHCH₂CH₃,
G-NH(CH₂)₃C(O)OCH₂CH₃,
G-NH(CH₂)₃C(O)NHCH₃,
G-NH(CH₂)₂C(O)OH,
G-NH(CH₂)₃C(O)OH,
Q-CH₂CH(CH₂(C₆H₁₁))C(O)NH(CH₂)₂C(O)OH,
Q-CH₂CH(CH₂(C₆H₁₁))C(O)NH(CH₂)₂C(O)NH₂,
Z-NHCH₂C(O)OH,
Z-NHCH₂C(O)NH₂,
Z-NHCH₂C(O)N(CH₃)₂,
Z-NHCH₂C(O)NHCH(CH₃)₂,
Z-NH(CH₂)₂C(O)OCH₂(C₆H₅),
Z-NH(CH₂)₂C(O)NHCH₂CH₃,
Z-NH(CH₂)₃C(O)NHCH₃,
Z-NHCH₂C(O)NHCH₂C(O)OH,
Z-NHCH₂C(O)OCH₂C(O)OCH₃,
Z-NHCH₂C(O)OCH₂C(O)NHCH₃,
Z-NHCH₂C(O)O-(4-methoxycyclohexyl),
Z-NHCH₂C(O)OCH₂C(O)NHCH₂(C₆H₅) and
Z-NHCH₂C(O)OCH(CH₃)OC(O)CH₃;
wherein:
Q represents 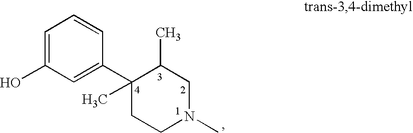
G represents 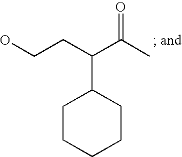
Z represents

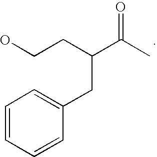

33. A method according to claim 32,
wherein said compound is selected from the group consisting of:
(+)-Z-NHCH₂C(O)OH,
(−)-Z-NHCH₂C(O)OH,
(3R,4R)-Z-NHCH₂C(O)NHCH₂(C₆H₅) and
(3R,4R)-G-NH(CH₂)₃C(O)OH.

34. A method according to claim 33,
wherein said compound is selected from the group consisting of:
(+)-Z-NHCH₂C(O)OH, and
(−)-Z-NHCH₂C(O)OH.

35. A method according to claim 34,
wherein said compound is selected from the group consisting of:
(+)-Z-NHCH₂C(O)OH.

36. A method according to claim 33,
wherein said compound is Q-CH₂CH(CH₂(C₆H₅))C(O)OH.

37. A method according to claim 36,
wherein said compound is (3R,4R,S)-Q-CH₂CH(CH₂(C₆H₅))C(O)OH.

38. A method according to claim 13,
wherein said compound is a substantially pure stereoisomer.

39. A method according to claim 1 or claim 13,
wherein the dose level of said 4-aryl-piperidine derivative or a stereoisomer, a prodrug, a pharmaceutically acceptable salt, a hydrate, a solvate, an acid salt hydrate, an N-oxide or an isomorphic form thereof is stepped up to the final dosage level over about 1 day to about 3 days.

40. A method according to claim 39,
wherein the dose level of said 4-aryl-piperidine derivative or a stereoisomer, a prodrug, a pharmaceutically acceptable salt, a hydrate, a solvate, an acid salt hydrate, an N-oxide or an isomorphic form thereof is stepped up to the final dosage level over about 3 days.

41. A method according to claim 39,
wherein said patient experiences fewer, less, or fewer and less adverse events associated with the administration of said 4-aryl-piperidine derivative or a stereoisomer, a prodrug, a pharmaceutically acceptable salt, a hydrate, a solvate, an acid salt hydrate, an N-oxide or an isomorphic form thereof; and
wherein said adverse events are selected from the group consisting of nausea, abdominal pain, vomiting, diarrhea, flatulence, abdominal distension, and combinations thereof.
